(19) United States
(12) Patent Application Publication
(10) Pub. No.: US 2022/0228111 A1
(43) Pub. Date: Jul. 21, 2022

(54) NOCICEPTOR DIFFERENTIATION FROM HUMAN PLURIPOTENT STEM CELLS

(71) Applicants: Ilyas SINGEC, Rockville, MD (US); Tao DENG, Rockville, MD (US); Anton SIMEONOV, Rockville, MD (US)

(72) Inventors: Ilyas Singec, Rockville, MD (US); Tao Deng, Rockville, MD (US); Anton Simeonov, Rockville, MD (US)

(21) Appl. No.: 17/605,876

(22) PCT Filed: Apr. 24, 2020
(86) PCT No.: PCT/US2020/029721

§ 371 Date: Oct. 22, 2021

Related U.S. Application Data
(60) Provisional application No. 62/837,891, filed on Apr. 24, 2019.

(51) Int. Cl. C12N 5/0793; A01N 1/02

(52) U.S. Cl. CPC C12N 5/062; A01N 1/0284; C12N 2501/13; C12N 2500/38; C12N 2506/45

(57) ABSTRACT

Methods for Generating Neural Crest-Like Cells and Nociceptor-Like Cells from Human Pluripotent Stem Cells are Provided Along with the Related Compositions.

### PRIOR RELATED APPLICATION

The present application claims the benefit of priority of U.S. Provisional Application No. 62/837,891, filed Apr. 24, 2019, which is incorporated herein by reference in its entirety.

### GOVERNMENT LICENSE RIGHTS

This invention was made with Government support awarded by NIH Regenerative Medicine Program of the National Institutes of Health (NIH Common Fund), National Center for Advancing Translational Sciences (NCATS). The Government has certain rights in the invention.

### FIELD

The present invention lies in the fields of biochemistry, cell biology, bioengineering, drug development and stem cell biology, as well as related fields, and concerns the compositions and methods useful for culturing and differentiating pluripotent stem cells.

### BACKGROUND

Pluripotency is a remarkable cellular state that allows differentiation of stem cells into any cell type of the human body. Vertebrate pluripotent stem cells, including embryonic stem cells (ESCs) and induced pluripotent stem cells (iPSCs), undergo extensive self-renewal and have the potential to differentiate into all somatic cell types. Generating desired cell types from pluripotent stem cells hold enormous potential for drug discovery, disease modeling and regenerative medicine. For instance, development of new non-addictive pain drugs for human use as well as for addiction research would greatly benefit from directed differentiation of human pluripotent stem cells (hPSCs) into relevant cells of the nervous system, such as nociceptors (also known as sensory neurons). Unfortunately, existing procedures of producing nociceptors from vertebrate pluripotent stem cells can be inefficient, undefined and lengthy. They also show poor reproducibility, require expensive supplements and often generate chaotic mixtures of different cell lineages. Therefore, a need exists for improved methods for generating cells exhibiting at least some characteristics of nociceptor cells from vertebrate pluripotent stem cells.

### SUMMARY

Described and included among the embodiments of the present invention are methods useful for production and maintenance in culture of differentiated vertebrate cells exhibiting at least some characteristics of vertebrate peripheral sensory neurons, such as nociceptor cells. Also described and included among the embodiments of the present invention are methods useful for production and maintenance in culture of vertebrate cells exhibiting at least some characteristics of neural crest cells. Among other things, the methods described in this document are highly efficient, cost-effective, reproducible, scalable and suitable for automation. The methods described in this document are useful, among other things, for example, in drug discovery and development, such as discovery and development of new pain medications, in nociceptor-related research, such as pain and addiction research, in high-throughput screening of compounds for various applications, including drug development and toxicity screening, in disease modeling and research, such as modeling and research of inherited and acquired neuropathies, as well as in regenerative therapies, such as cell replacement and repair of damaged nerves, and cell and tissue engineering.

The advantages of the compositions, kits and methods of the present invention are discussed throughout this document and illustrated in the accompanying figures.

The terms “invention,” “the invention,” “this invention” and “the present invention,” as used in this document, are intended to refer broadly to all of the subject matter of this patent application and the claims below. Statements containing these terms should be understood not to limit the subject matter described herein or to limit the meaning or scope of the patent claims below. Covered embodiments of the invention are defined by the claims, not this summary. This summary is a high-level overview of various aspects of the invention and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim. The present document describes and refers to various embodiments of the invention. No particular embodiment is intended to define the scope of the invention. Rather, the embodiments merely provide non-limiting examples of various methods, compositions, kits, systems etc. that are at least included within the scope of the invention. Some embodiments of the present invention are summarized below, while others are described and shown elsewhere in the present document.

Exemplary embodiments of the present invention include methods of producing in culture cells capable of differentiating into nociceptor-like cells. Some methods of producing in culture cells capable of differentiating into nociceptor-like cells according to the embodiments of the present invention comprise the steps of: incubating for approximately 24-144 hours attached monolayer cultures of vertebrate pluripotent stem cells in a first medium comprising an effective amount or concentration of at least one first compound capable of activating WNT signaling and an effective concentration of at least one second compound capable of inhibiting TGF-beta signaling; dissociating the incubated cells; and, culturing the dissociated cells for 168-432 hours in a second medium comprising an effective amount or concentration of at least one third compound capable of activating WNT signaling, an effective amount or concentration of at least one fourth compound capable of inhibiting TGF-beta signaling, an effective amount or concentration of at least one fifth compound capable of inhibiting Notch pathway, and an effective concentration of at least one sixth compound capable of inhibiting one or more of EGF, VEGF and MAP kinase signaling, thereby generating one or more nocispheres comprising the cells capable of differentiating into the nociceptor-like cells. In some embodiments, the cells capable of differentiating into nociceptor-like cells are neural crest-like cells. In some embodiments, the cells capable of differentiating into nociceptor-like cells detectably express SOX10. In some embodiments, one or more nocispheres further comprises the nociceptor-like cells. In some embodiments, the nociceptor-like cells detectably express BRN3A. In some embodiments of the methods, the vertebrate pluripotent stem cells are induced pluripotent stem cells or embryonic pluripotent stem cells. In some embodiments, the vertebrate pluripotent stem cells are human pluripotent stem cells. In some embodiments, the first medium is a defined medium and the second medium is a defined medium, and the first medium is the same or different from the second medium. The first medium can be E6, DMEM-F12 or Knockout-DMEM/F12. The second medium can be E6, DMEM-F12 or Knockout-DMEM/F12. In some embodiments, the first medium and/or the second medium is not supplemented with additives activating or inhibiting bone morphogenic protein (BMP) protein pathways. In some embodiments, the first medium and/or the second medium is not supplemented with Bone Morphogenic Protein 4 (BMP4). In some embodiments, the at least one first compound is CHIR98014 or CHIR99021, or a combination thereof. In some embodiments, the effective concentration of CHIR98014 or CHIR99021 is 20 nM-20 μM. In some embodiments, the at least one second compound is A83-01 or SB431542, or a combination thereof. In some embodiments, the effective concentration of A83-01 is 20 nM-20 μM, and the effective concentration of SB431542 is 20 nM-40 μM. In some embodiments, the at least one third compound is CHIR98014 or CHIR99021, or a combination thereof. In some embodiments, the effective concentration of CHIR98014 or CHIR99021 is 20 nM-20 μM. In some embodiments, the at least one fourth compound is A83-01 or SB431542, or a combination thereof. In some embodiments, the effective concentration of A83-01 is 20 nM-20 μM, and the effective concentration of SB431542 is 20 nM-40 μM. In some embodiments, the at least one fifth compound is DBZ, DAPT, LY411575 or LY 3039478, or a combination of two or more thereof. In some embodiments, the effective concentration of DBZ is 20 nM-20 μM, the effective concentration of DAPT is 5 nM-50 μM, the effective concentration of LY411575 is 2 nM-20 μM, and the effective concentration of LY3039478 is 2 nM-20 μM. In some embodiments, the at least one sixth compound is PD173074 or SU5402, or a combination thereof. In some embodiments, the effective concentration of PD173074 or SU5402 is 2 nM-20 μM. In some embodiments, the second medium further comprises an effective amount or concentration of at least one seventh compound that is a CDK4/6 inhibitor. In some embodiments, the CDK4/6 inhibitor is PD0332991. In some embodiments, the effective concentration of PD0332991 is 2 nM-20 μM. In some embodiments, the step of culturing of the dissociated cells comprises changing the second medium changing approximately every 12-36 hours. Some embodiments of the above methods further comprise a step of dissociating the one or more nocispheres, thereby generating dissociated nocisphere cells. Some embodiments of the above methods further comprise a step of cryopreserving one or more of the cells capable of differentiating into the nociceptor-like cells, the one or more nocispheres, or the dissociated nocisphere cells. In some embodiments of the above methods, one or more steps of the method is performed by an automated system.

Exemplary embodiments of the present invention include methods of culturing nociceptor-like cells, comprising the steps of: incubating for approximately 24-144 hours attached monolayer cultures of vertebrate pluripotent stem cells in a first medium comprising an effective amount or concentration of at least one first compound capable of activating WNT signaling and an effective concentration of at least one second compound capable of inhibiting TGF-beta signaling; dissociating the incubated cells; culturing the dissociated cells for 168-432 hours in a second medium comprising an effective amount or concentration of at least one third compound capable of activating WNT signaling, an effective amount or concentration of at least one fourth compound capable of inhibiting TGF-beta signaling, an effective amount or concentration of at least one fifth compound capable of inhibiting Notch pathway, and an effective concentration of at least one sixth compound capable of inhibiting one or more of EGF, VEGF and MAP kinase signaling, thereby generating one or more nocispheres comprising the cells capable of differentiating into the nociceptor-like cells; dissociating the one or more nocispheres, thereby generating dissociated nocisphere cells, and, growing in culture the dissociated nocisphere cells under conditions promoting differentiation of the nociceptor-like cells. In some embodiments, the cells capable of differentiating into nociceptor-like cells are neural crest-like cells. In some embodiments, the cells capable of differentiating into nociceptor-like cells detectably express SOX10. In some embodiments, one or more nocisphere further comprises the nociceptor-like cells. In some embodiments, the nociceptor-like cells detectably express BRN3A. In some embodiments of the methods, the vertebrate pluripotent stem cells are induced pluripotent stem cells or embryonic pluripotent stem cells. In some embodiments, the vertebrate pluripotent stem cells are human pluripotent stem cells. In some embodiments, the first medium is a defined medium and the second medium is a defined medium, and the first medium is the same or different from the second medium. The first medium can be E6, DMEM-F12 or Knockout-DMEM/F12. The second medium can be E6, DMEM-F12 or Knockout-DMEM/F12. In some embodiments, the first medium and/or the second medium is not supplemented with additives activating or inhibiting bone morphogenic protein (BMP) protein pathways. In some embodiments, the first medium and/or the second medium is not supplemented with Bone Morphogenic Protein 4 (BMP4). In some embodiments, the at least one first compound is CHIR98014 or CHIR99021, or a combination thereof. In some embodiments, the effective concentration of CHIR98014 or CHIR99021 is 20 nM-20 μM. In some embodiments, the at least one second compound is A83-01 or SB431542, or a combination thereof. In some embodiments, the effective concentration of A83-01 is 20 nM-20 μM, and the effective concentration of SB431542 is 20 nM-40 μM. In some embodiments, the at least one third compound is CHIR98014 or CHIR99021, or a combination thereof. In some embodiments, the effective concentration of CHIR98014 or CHIR99021 is 20 nM-20 μM. In some embodiments, the at least one fourth compound is A83-01 or SB431542, or a combination thereof. In some embodiments, the effective concentration of A83-01 is 20 nM-20 μM, and the effective concentration of SB431542 is 20 nM-40 μM. In some embodiments, the at least one fifth compound is DBZ, DAPT, LY411575 or LY 3039478, or a combination of two or more thereof. In some embodiments, the effective concentration of DBZ is 20 nM-20 μM, the effective concentration of DAPT is 5 nM-50 μM, the effective concentration of LY411575 is 2 nM-20 μM, and the effective concentration of LY3039478 is 2 nM-20 μM. In some embodiments, the at least one sixth compound is PD173074 or SU5402, or a combination thereof. In some embodiments, the effective concentration of PD173074 or SU5402 is 2 nM-20 μM. In some embodiments, the second medium further comprises an effective amount or concentration of at least one seventh compound that is a CDK4/6 inhibitor. In some embodiments, the CDK4/6 inhibitor is PD0332991. In some embodiments, the effective concentration of PD0332991 is 2 nM-20 μM. In some embodiments, the step of culturing of the dissociated cells comprises changing the second medium approximately every 12-36 hours. In some embodiments, the step of growing in culture the dissociated nocisphere cells is conducted for at least approximately 168 hours or for 168-336 hours. In some embodiments, the conditions promoting differentiation of the nociceptor-like cells comprise having the presence of N2 supplement and B27 supplement, and can further comprise having the presence of one or more of BDNF, GDNF, NGF or NT-3. In some embodiments, the conditions promoting differentiation of the nociceptor-like cells can further comprise the presence of one or more of an effective amount or concentration at least one eighth compound capable of inhibiting Notch pathway, an effective amount or concentration of at least one ninth compound capable of inhibiting one or more of EGF, VEGF and MAP kinase signaling or an effective amount or concentration at least one ninth compound that is a CDK4/6 inhibitor. In some embodiments, the at least one eighth compound is DBZ, DAPT, LY411575 or LY 3039478, or a combination of two or more thereof. In some embodiments, the effective concentration of DBZ is 20 nM-20 μM, the effective concentration of DAPT is 5 nM-50 μM, the effective concentration of LY411575 is 2 nM-20 μM, and the effective concentration of LY3039478 is 2 nM-20 μM. In some embodiments, the at least one sixth compound is PD173074 or SU5402, or a combination thereof. In some embodiments, the effective concentration of PD173074 or SU5402 is 2 nM-20 μM. In some embodiments, the CDK4/6 inhibitor is PD0332991. In some embodiments, the effective concentration of PD0332991 is 2 nM-20 μM. In some embodiments of the above methods, the conditions promoting differentiation of the nociceptor-like cells comprise the absence of supplementation with additives activating or inhibiting Bone Morphogenic Protein (BMP) protein pathways. In some embodiments, the conditions comprise absence of supplementation with Bone Morphogenic Protein 4 (BMP4). In some embodiments, the conditions promoting differentiation of the nociceptor-like cells comprise culturing in DMEM/F12 medium, Neurobasal medium or BrainPhys medium. In some embodiments of the above methods, the nociceptor-like cells detectably express one or more of TUJ1, Peripherin, ISL1, GGRP, TRPV1, NAV1.7, NAV1.8, NAV1.9, OPRM1, OPRMK1, OPRD1, OPRL1 or NF200. For example, the nociceptor-like cells can detectably express NAV1.8, OPRM1, OPRMK1 and OPRD1. In some embodiments, the nociceptor-like cells lack dendrites detectably expressing MAP2. Some embodiments of the above methods further comprise a step of cryopreserving one or more of the cells capable of differentiating into the nociceptor-like cells, the one or more nocispheres, or the dissociated nocisphere cells. Some embodiments of the above methods comprise, after the step of dissociating the one or more nocispheres, cryopreserving the dissociated nocisphere cells and thawing of the dissociated nocisphere cells. Some embodiments of the above methods comprise a step of cryopreserving the nociceptor-like cells. In some embodiments, the one or more steps of cryopreserving the one or nocispheres, cryopreserving the dissociated nocisphere cells or cryopreserving of the nociceptor-like cells is conducted in a cryopreservation medium comprising Chroman 1 and/or a derivative thereof, Emricasan and/or the derivative thereof, trans-ISRIB and polyamines comprising putrescine, spermine and spermidine. In some embodiments, in the cryopreservation medium, Chroman 1 and/or the derivative thereof is at a concentration of about 4 nM to about 40 μM, about 10 nM to about 20 μM, about 20 nM to about 10 μM or about 30 nM to about 500 nM, Emricasan and/or the derivative thereof at a concentration of about 100 nM to about 40 μM, about 200 nM to about 30 μM, about 300 nm to about 20 μM, trans-ISRIB at a concentration of about 50 nM to about 6.25 μM, about 100 nM to about 6.25 μM, or about 200 nM to about 6.25 μM, and putrescine, spermine and spermidine is each at a concentration of about 0.5 μM to 1 mM. In some embodiments of the above methods, one or more steps of the method is performed by an automated system.

Exemplary embodiments of the present invention include compositions comprising at least one cultured nociceptor-like cell detectably expressing one or more of BRN3A, TUJ1, Peripherin, I1, GGRP, TRPV1, NAV1.7, NAV1.8, NAV1.9, OPRM1, OPRMK1, OPRD1, OPRL1 or NF200. In some embodiments, at least one cultured nociceptor-like cell detectably expresses NAV1.8, OPRM1, OPRK1 and OPRD1. In some embodiments, the at least one cultured nociceptor-like cell lacks dendrites detectably expressing MAP2. In some embodiments, the at least one cultured nociceptor-like cell is or has been cryopreserved. In some embodiments, the at least one cultured nociceptor-like cell is or was cryopreserved in a cryopreservation medium comprising Chroman 1 and/or a derivative thereof, Emricasan and/or the derivative thereof, trans-ISRIB and polyamines comprising putrescine, spermine and spermidine. In some embodiments, in the cryopreservation medium, Chroman 1 and/or the derivative thereof is at a concentration of about 4 nM to about 40 μM, about 10 nM to about 20 μM, about 20 nM to about 10 μM or about 30 nm to about 500 nM, Emricasan and/or the derivative thereof at a concentration of about 100 nM to about 40 μM, about 200 nm to about 30 μM, about 300 nM to about 20 μM, trans-ISRIB at a concentration of about 50 nM to about 6.25 μM, about 100 nM to about 6.25 μM, or about 200 nM to about 6.25 μM, and putrescine, spermine and spermidine is each at a concentration of about 0.5 μM to 1 mM. Exemplary embodiments of the present invention also include cell cultures comprising the above compositions. Cell cultures according to the embodiments of the present invention can further comprise a culture medium comprising Chroman 1 and/or a derivative thereof, Emricasan and/or the derivative thereof, trans-ISRIB and polyamines comprising putrescine, spermine and spermidine. In some embodiments, in the culture medium, Chroman 1 and/or the derivative thereof is at a concentration of about 4 nM to about 40 μM, about 10 nM to about 20 μM, about 20 nM to about 10 μM or about 30 nm to about 500 nM, Emricasan and/or the derivative thereof at a concentration of about 100 nM to about 40 μM, about 200 nM to about 30 μM, about 300 nM to about 20 μM, trans-ISRIB at a concentration of about 50 nM to about 6.25 μM, about 100 nM to about 6.25 μM, or about 200 nM to about 6.25 μM, and putrescine, spermine and spermidine is each at a concentration of about 0.5 μM to 1 mM. In some embodiments, the cell culture is grown from previously cryopreserved cells. In some embodiments, the previously cryopreserved cells were cryopreserved in a cryopreservation medium comprising Chroman 1 and/or a derivative thereof, Emricasan and/or the derivative thereof, trans-ISRIB and polyamines comprising putrescine, spermine and spermidine. In some embodiments, the cryopreservation medium comprises Chroman 1 and/or the derivative thereof at a concentration of about 4 nM to about 40 μM, about 10 nM to about 20 μM, about 20 nM to about 10 μM or about 30 nM to about 500 nM, Emricasan and/or the derivative thereof at a concentration of about 100 nM to about 40 μM, about 200 nM to about 30 μM, about 300 nM to about 20 μM, trans-ISRIB at a concentration of about 50 nM to about 6.25 μM, about 100 nM to about 6.25 μM, or about 200 nM to about 6.25 μM, and putrescine, spermine and spermidine is each at a concentration of about 0.5 μM to 1 mM.

Exemplary embodiments of the present invention include compositions comprising at least one cultured neural crest-like cell. In some embodiments, the at least one cultured neural crest-like cell is capable of differentiating into at least one nociceptor-like cell. In some embodiments, the at least one cultured neural crest-like cell detectably expresses SOX10. Some embodiments of the above compositions further comprise at least one cultured nociceptor-like cell detectably expressing one or more of BRN3A, TUJ1, Peripherin, GGRP, TRPV1, NAV1.7, NAV1.8, NAV1.9, OPRM1, OPRMK1, OPRD1, OPRL1 or NF200. In some embodiments, the at least one cultured nociceptor-like cell detectably expresses NAV1.8, OPRM1, OPRK1 and OPRD1. In some embodiments, the at least one cultured nociceptor-like cell lacks dendrites detectably expressing MAP2. In some embodiments, the composition comprising at least one cultured neural crest-like cell is or comprises one or more nocispheres. In some embodiments, the composition comprises dissociated nocisphere cells. In some embodiments, the composition comprising at least one cultured neural crest-like cell is or was cryopreserved. In some embodiments, the composition is or was cryopreserved in a cryopreservation medium comprising Chroman 1 and/or a derivative thereof, Emricasan and/or the derivative thereof, trans-ISRIB and polyamines comprising putrescine, spermine and spermidine. In some embodiments, in the cryopreservation medium, Chroman 1 and/or the derivative thereof is at a concentration of about 4 nM to about 40 about 10 nM to about 20 μM, about 20 nM to about 10 μM or about 30 nM to about 500 nM, Emricasan and/or the derivative thereof at a concentration of about 100 nM to about 40 about 200 nM to about 30 μM, about 300 nM to about 20 trans-ISRIB at a concentration of about 50 nM to about 6.25 about 100 nM to about 6.25 or about 200 nM to about 6.25 and putrescine, spermine and spermidine is each at a concentration of about 0.5 μM to 1 mM. Exemplary embodiments of the present invention also include cell cultures comprising the composition comprising at least one cultured neural crest-like cell. In some embodiments, the cell cultures further comprising a culture medium comprising Chroman 1 and/or a derivative thereof, Emricasan and/or the derivative thereof, trans-ISRIB and polyamines comprising putrescine, spermine and spermidine. In some embodiments, in the culture medium, Chroman 1 and/or the derivative thereof is at a concentration of about 4 nM to about 40 about 10 nM to about 20 μM, about 20 nM to about 10 μM or about 30 nM to about 500 nM, Emricasan and/or the derivative thereof at a concentration of about 100 nM to about 40 about 200 nM to about about 300 nm to about 20 trans-ISRIB at a concentration of about 50 nM to about 6.25 about 100 nM to about 6.25 or about 200 nM to about 6.25 and putrescine, spermine and spermidine is each at a concentration of about 0.5 μM to 1 mM. In some embodiments, the cell culture is grown from previously cryopreserved cells. In some embodiments, the previously cryopreserved cells were cryopreserved in a cryopreservation medium comprising Chroman 1 and/or a derivative thereof, Emricasan and/or the derivative thereof, trans-ISRIB and polyamines comprising putrescine, spermine and spermidine. In some embodiments, in the cryopreservation medium Chroman 1 and/or the derivative thereof is at a concentration of about 4 nM to about 40 about 10 nM to about 20 about 20 nM to about 10 μM or about 30 nM to about 500 nM, Emricasan and/or the derivative thereof at a concentration of about 100 nM to about 40 about 200 nM to about 30 about 300 nM to about 20 trans-ISRIB at a concentration of about 50 nM to about 6.25 about 100 nM to about 6.25 or about 200 nM to about 6.25 and putrescine, spermine and spermidine is each at a concentration of about 0.5 μM to 1 mM.

### BRIEF DESCRIPTION OF THE FIGURES

FIG. 1 is a schematic illustration of an exemplary procedure for producing nociceptor-like cells.

FIG. 2 depicts structures of exemplary small molecules used in some of the embodiments of the present invention.

FIG. 3 shows representative images of plated cells taken at day 28 of culturing according to an exemplary procedure for producing nociceptor-like cells. The cells were immunocytochemically stained with the indicated combinations of antibodies specific for the following proteins: TUJ1 (also known as beta-III Tubulin, neuronal marker); BRN3A (transcription factor typically expressed by nociceptors); peripherin (PRPH, marker for peripheral neurons); ISL1 (transcription factor expressed by nociceptors); CGRP (calcitonin-gene related protein, neuropeptide typically expressed by nociceptors); TRPV1 (vanilloid receptor 1, typically expressed by nociceptors); NAV1.7 (sodium channel, typically expressed by nociceptors). The label “Ho” indicates Hoechst counterstain, which labels cell nuclei.

FIG. 4A shows representative images of plated cells taken at day 28 of culturing according to an exemplary procedure for producing nociceptor-like cells. The cells were immunocytochemically stained with specific antibodies against NF200 (neurofilament 200, typical marker to visualize axons) and transcription factor BRN3A (transcription factor typically expressed by nociceptors). Hoechst dye was used as a counterstain to visualize cell nuclei. The merged image in the bottom right corner of FIG. 4A shows all the stains together.

FIG. 4B shows representative images of plated cells taken at day 28 of culturing according to the exemplary procedure for producing nociceptor-like cells. The cells were immunocytochemically stained with specific antibodies against TUJ1 and vGLUT1 (vesicular glutamate transporter 1, a marker for glutamatergic neurons) confirming the expected glutamatergic neurotransmitter phenotype of nociceptor-like cells. Hoechst dye was used as a counterstain to visualize cell nuclei. The merged image in the bottom right corner of FIG. 4B shows all the stains together.

FIG. 4C shows representative images of cells at day 28 cultured according to an exemplary procedure for producing nociceptor-like cells. The cells were immunocytochemically stained with specific antibodies against MAP2 (microtubule associated protein 2, a typical marker for neuronal cell bodies and dendrites, which only labels cell bodies of pseudounipolar nociceptor-like cells lacking dendrites similar to their in vivo counterparts) and the transcription factor BRN3A (transcription factor typically expressed by nociceptors). Hoechst dye was used as a counterstain to visualize cell nuclei. The merged image in the bottom right corner of FIG. 4C shows all the stains together.

FIG. 5 shows a bar graph illustrating quantitative characterization of the plated cells at day 28 of culturing according to an exemplary procedure for producing nociceptor-like cells. Two hPSC lines, hESCs and iPSCs, were differentiated into nociceptor-like cells. At day 28, the cells were stained for SOX10 (marker for neural crest stem cells) and BRN3A (marker for nociceptors), and the staining was quantified.

FIG. 6 illustrates the results of systematic analysis of the time course of gene expression over days 0-28 in the iPSCs subjected to an exemplary procedure for producing nociceptor-like cells.

FIG. 7 illustrates the results of time-course gene expression profiling by RNA-seq of the cells differentiated according to the procedure for producing nociceptor-like cells and comparison of the results to those available in ARCHS⁴ human tissue RNA-seq database.

FIG. 8 illustrates the results of comprehensive analysis by RNA-seq of the ion channels and receptors expressed by nociceptor-like cells.

FIG. 9 shows the line plots illustrating the results of time-course gene expression profiling by RNA-seq of important sodium channels.

FIG. 10 shows the line plots illustrating the results of time-course gene expression profiling by RNA-seq of opioid receptors (OPRM1, OPRK1, OPRD1) and opioid related nociceptin receptor 1 (OPRL1) in the cultures being differentiated from iPSCs.

FIG. 11 illustrates the results of an electrophysiology experiment (multi-electrode array) demonstrating that iPSC-derived nociceptor-like cells were stimulated by application of DMSO, 10 μM α,β-me-ATP, 5 μM capsaicin, and 100 μM mustard oil (allyl isothiocyanate).

FIG. 12A illustrates the results of an electrophysiology experiment (multi-electrode array) showing that iPSC-derived nociceptor-like cells were sensitized in response to treatment with oxaliplatin and prostaglandin E2 (PGE2).

FIG. 12B is a bar graph illustrating quantitative analysis of the experimental results illustrated in FIG. 12A.

FIG. 13 illustrates the results of electrophysiology experiments (multi-electrode array) demonstrating that iPSC-derived nociceptor-like cells were stimulated by application of 10 μM α,β-me-ATP. Differential response of iPSC-derived nociceptor-like cells to specific inhibitors of the purinergic receptor P2RX3 was also tested, identifying RO-51 as the most potent inhibitor blocking the effect of 10 μM α,β-me-ATP.

FIG. 14A shows a photograph of CompacT SelecT® system (Sartorius) used for performing an automated exemplary procedure for producing nociceptor-like cells.

FIG. 14B is a representative microscopic image of nociceptor-like cells produced by an exemplary automated procedure for producing nociceptor-like cells. The representative image was acquired at day 21 after starting the differentiation procedure.

### DESCRIPTION

The embodiments of the present invention were envisioned at least in part based on the discoveries discussed below. By manipulating critical cell signaling pathways at defined timepoints by using small molecule inhibitors, the inventors discovered a procedure for converting human pluripotent stem cells in culture into SOX10-expressing cells resembling neural crest cells. The SOX10-expressing cells were subjected to a differentiation procedure and produced, in a highly reproducible fashion, a homogenous population of BRN3A-expressing cells resembling nociceptors. Extensive morphological, molecular and electrophysiological characterization experiments confirmed nociceptor-like properties of the resulting differentiated cells, including expression of typical nociceptor markers, such as neuronal and synaptic proteins, transcription factors, neuropeptides and over 150 ion channels. Of particular interest for pain research and drug development applications, nociceptor-like cells generated with the methods discovered by inventors expressed complex ion channels and receptor molecules known to be present in naturally occurring nociceptors, such as NAV1.8, opioid receptors and purinergic receptors such as P2RX3. Previously known stem cell differentiation methods were unable to generate the cells with the above properties. Using a robotic cell culture system, the inventors automated the procedure for generating nociceptor-like cells from human pluripotent stem cells. Based on the above discoveries, the inventors conceived processes (methods) for producing in culture cells capable of differentiating into cells exhibiting at least some characteristics of vertebrate nociceptor cells, including human nociceptor cells, processes (methods) of producing in culture of cells exhibiting at least some characteristics of vertebrate neural crest cells, including human nociceptor cells, as well as various compositions and kits related to the above processes. The inventors also conceived various applications and uses of their processes (methods), compositions and kits, including high-throughput applications and uses requiring large numbers of standardized cells of high quality. Among other things, various embodiments of the invention described in this document can be used in drug discovery and development, toxicity screenings, disease modeling and research, cell and tissue engineering, cell replacement therapies and regenerative medicine.

### Terms and Concepts

A number of terms and concepts are discussed below. They are intended to facilitate the understanding of various embodiments of the invention in conjunction with the rest of the present document and the accompanying figures. These terms and concepts may be further clarified and understood based on the accepted conventions in the fields of the present invention and the description provided throughout the present document and/or the accompanying figures. Some other terms can be explicitly or implicitly defined in other sections of this document and in the accompanying figures and may be used and understood based on the accepted conventions in the fields of the present invention, the description provided throughout the present document and/or the accompanying figures. The terms not explicitly defined can also be defined and understood based on the accepted conventions in the fields of the present invention and interpreted in the context of the present document and/or the accompanying figures.

As used herein, the terms “a,” “an,” and “the” can refer to “one,” “one or more” or “at least one,” unless specifically noted otherwise.

The terms “about” or “approximately” are used herein to indicate that a value includes the inherent variation of error for the device, the method being employed to determine the value, or simply error-tolerance of a value. For example, the term “about” may mean±1%, ±5%, ±10%, ±15% or ±20% variation from a predetermined value.

As used herein, the terms “isolate,” “separate” or “purify” and the related terms are not used necessarily to refer to the removal of all materials other than the components of interest from a sample. Instead, in some embodiments, the terms are used to refer to a procedure that enriches the amount of one or more components of interest relative to one or more other components present in the sample. In some embodiments, “isolation,” “separation” or “purification” may be used to remove or decrease the amount of one or more components from a sample. For example, the expression “an isolated cell” can refer to a cell that has been substantially separated or purified away from other cells of a cell culture or an organism.

The term “derived” and the related expressions referring to cells or a biological sample indicate that the cell or sample was obtained from the stated source at some point in time. For example, a cell derived from an organism can represent a primary cell obtained directly from the individual (that is, unmodified), or it can be modified, for example, by introduction of a recombinant vector, by exposure to or culturing under particular conditions, or immortalization. In some cases, a cell derived from a given source will undergo cell division and/or differentiation such that the original cell is no longer exists, but the continuing cells will be understood to derive from the same source. The term “derive,” “derivation” and the related terms and expressions can also be used in this document to refer to creation of a cell population from a different starting or preceding population or cell. For example, in cases of a population of differentiated nociceptor-like cells described in this document, the starting populations are neural crest-like cells and pluripotent stem cells. Thus, nociceptor-like cells can be described as being derived from neural crest-like cells and/or pluripotent stem cell or cells. In another example, a population of neural crest-like cells described in this document, the starting population is pluripotent stem cell or cells. Thus, neural crest-like cells can be described as being derived from pluripotent stem cell or cells.

The term “comprising” and the related terms (“comprise,” “comprises,” etc.), when used in this document to describe various embodiments of the invention, are open-ended, meaning that they do not exclude additional elements and synonymous with terms “including,” “containing” or “having.” When an embodiment of the invention is described using the term “comprising,” it is intended to include the embodiments, in which the term comprising is replaced with the terms “consisting of” or “consisting essentially of” In other words, the description of the embodiments of the invention described in this document using the term “comprising” and the related terms also provides the description of the related embodiments that use “consisting of” or “consisting essentially of” instead of “comprising”. The term “consisting of” excludes any elements (steps, ingredient etc.) not specified in the description. The term “consisting essentially of” is intended to exclude only those elements not specified in the description that do not materially affect the basic and novel characteristics of the embodiment.

The terms “culture,” “cell culture” and related terms can be used to refer to a cell or a population of cells residing outside of an organism. These cells can be stem cells, primary cells isolated from an organism or obtained from a cell bank, animal, or blood bank, or secondary cells that are derived from such sources. Secondary cells can be immortalized for long-lived cell culture. A primary cell includes any cell of an adult or fetal organism apart from egg cells, sperm cells and stem cells. Examples of useful primary cells include, but are not limited to, skin cells, bone cells, blood cells, cells of internal organs and cells of connective tissue. A secondary cell is derived from a primary cell and can be immortalized for long-lived in vitro cell culture.

The terms “culture,” “culturing,” “grow,” “growing,” “maintain,” “maintaining,” “expand,” “expanding,” etc., when referring to cell, tissue or organ culture or the process of culturing, can be used interchangeably to mean that a cell or a group of cells (the scope of which expression includes groups or pluralities of undifferentiated or differentiated cells, embryos, embryoid bodes, tissues or organs) is maintained outside the body (ex vivo and/or in vitro) under conditions suitable for survival, proliferation, differentiation and/or avoiding senescence. In other words, cultured cell or groups of cells are allowed to survive, and culturing can result in cell growth, differentiation, or division. In this context, the terms “growing” and “culturing” can be used interchangeably and can refer to maintaining living cells in culture under certain conditions. The terms above do not imply that all cells in the culture survive or grow or divide, as some may naturally senesce. Cells are typically cultured in media, which can be changed during the course of the culture. The so-called two-dimensional (2D) cell cultures grow on flat surfaces, typically in plastic vessels that can be coated with substrates (for example, vitronectin, laminin 521, Matrigel, Geltrex). Three-dimensional (3D) cultures are cultures in which biological cells are permitted to grow or interact with their surroundings in all three dimensions. 3D cultures can be grown in in a variety of artificial environments, such as, but not limited to, plates, flasks, bioreactors or small capsules in which the cells can grow into spheroids, spheres or nocispheres. 3D cultures include so-called scaffold-free and scaffold-based technologies. Scaffold-free methods employ, but are not limited to, the uses of low adhesion plates, hanging drop plates, micropatterned surfaces, and rotating bioreactors, magnetic levitation, and magnetic 3D bioprinting. Scaffolds are structures or materials that provide a structural support for cell attachment and, in some cases, differentiation. Scaffolds include solid scaffolds, sponges (such as cellulose sponges), protein-based scaffolds (such as collagen or gelatin-based scaffolds), hydrogels, nanofiber scaffolds, synthetic polymer scaffolds (for example, polycaprolactone or polystyrene scaffolds). In general, the culture environment includes consideration of such factors as the substrate for cell growth, cell density and cell contract, the gas phase, the medium, and temperature. Cells in culture are generally maintained under conditions known to be optimal for cell growth. Such conditions may include, for example, a temperature of approximately 37° C. and a humidified atmosphere containing approximately 5% CO₂. The duration of the incubation can vary widely, depending on the desired results.

The terms “medium,” “culture medium,” “culture solution,” “growth medium” and the related terms and expression refer to a medium supporting the survival and/or growth of cells (including single cells and pluralities of cells), tissues, organs or parts thereof or embryonic structures (such as, but not limited to, morula, blastocoel, blastocyst or embryo). A medium is typically isotonic, and can be a liquid, a colloidal liquid, a gel, a solid and/or a semi-solid. A medium can be configured to provide a matrix for cell adhesion or support, or a separate support (such as a culture vessel surface or a scaffold) can be provided. A medium can include the components for nutritional, chemical, and structural support necessary for culturing a cell or cells. A chemically defined medium (or “defined medium”) is a medium with known concentrations of all of its chemical components. In contrast, an undefined medium can contain complex biological components, such as serum albumin or serum, that do not have completely defined compositions. A conditioned medium is understood to be a previously used medium from cultured cells. It contains metabolites, growth factors, and extracellular matrix proteins secreted into the medium by the cultured cells, which can be beneficial for subsequent uses of such conditioned medium. Culture medium can be provided in a powdered form to be prepared prior to use, in a concentrated form to be diluted prior to use, or in a form to be used without further dilution. For example, a culture medium can be a sterile liquid, supplied as a “working solution” to be used without further dilution, in which case the culture medium. A working solution of culture medium can contain effective amounts or concentrations of one or more additives. In another example, a culture medium can be a gel containing effective amounts of one or more additives. When a culture medium is provided in a form requiring further preparation, such as a powder or a concentrate, one or more can be included in amounts or concentration intended to provide an effective amount or amounts after the medium is prepared. For example, a 2× concentrated medium may contain twice the effective amount or amounts of one or more additives intended to be included in the final “working” form of the medium. Culture medium typically contains one or more appropriate nutrient sources for growth and/or maintenance of cells it is intended to support, such as mammalian cells, including human cells. Culture medium maintains appropriate pH and osmolarity. Culture medium can contain natural ingredients, artificial ingredients and/or synthetic ingredients. Examples of natural ingredients are biological fluids (such as plasma, serum, lymph or amniotic fluid), tissue extracts (such as extracts of liver, spleen, tumors, leukocytes, bone marrow or animal embryos). Example of culture media composed of artificial ingredients (“artificial media”) are MEM and DMEM. Artificial culture medium can be serum-containing culture medium, serum-free culture medium (which can contain defined qualities of purified growth factors, lipoproteins and other components provided by the serum), chemically defined culture medium or protein-free culture medium. Culture medium can comprise one or more of a buffer, one or more inorganic salt, essential amino acids, one or more carbohydrate, such as glucose, fatty acids, lipids, vitamins and trace elements. One example of a buffer is a so-called natural buffering system, in which gaseous CO₂ balances with the CO₃^{2-/}HCO³⁻ content of the culture. Another example is a chemical buffering system, such as the one using 4-(2-hydroxyethyl)-1-piperazineethanesulfonic acid (HEPES), a zwitterionic buffering agent. Culture medium can contain a pH indicator, such as phenol red, which allows pH monitoring during cell growth. Inorganic salt or salts in the culture media supply sodium, potassium and calcium ions, provide osmotic balance and help regulating cell membrane potential. Essential amino acids, which cannot be synthesized by the cells, are included in the culture medium, but nonessential amino acids may also be included to improve cell growth and viability. Carbohydrates, such as glucose, galactose, maltose or fructose are included as a source of energy. Proteins and peptides, such as albumin, transferrin or fibronectin may also be included, as well as fatty acids and lipids, particularly in serum-free media. Vitamins essential for growth and proliferation of cells, such as B group vitamins, can also be included. Examples of trace elements added to culture media, particularly serum free media, are copper, zinc, and selenium. Some examples of the culture media are commercially available media, such as, but not limited to, Essential 8 Medium, CTS Essential 8 Medium, Essential 6 Medium, StemFlex Medium, CTS KnockOut SR Xeno-free Medium, KnockOut Serum Replacement, StemPro, mTeSR, mTeSR1, StemFit, Nutristem, L7 Medium, iPS-Brew, Neurobasal or BrainPhys.

In the context of cell culture, the term “dissociating” can refer to a process of isolating cells from other cells or from a surface, such as a culture plate surface. For example, cells can be dissociated from an organ or a tissue by mechanical or enzymatic methods. In another example, cells that aggregate in vitro can be dissociated from each other. In yet another example, adherent cells are dissociated from a culture plate or other surface. Dissociation can involve breaking cell interactions with extracellular matrix (ECM) and substrates (for example, culture surfaces) or breaking the ECM between cells.

A “stem cell” is a cell characterized by the ability of self-renewal through mitotic cell division and the potential to differentiate into a tissue or an organ. Among stem cells, embryonic and somatic stem cells may be distinguished. For example, mammalian embryonic stem cells may reside in the blastocyst and give rise to embryonic tissues, whereas somatic stem cells may reside in adult tissues for the purpose of tissue regeneration and repair.

The term “cell line” typically refers to a cell culture developed from a single cell of a multicellular organism. Cells of a cell line have a relatively uniform genetic makeup. Some cell lines originate from stem cells. Some cell lines originate from naturally occurring cancerous cells that underwent genetic modifications (such as one or more mutations or introductions of viral genes) leading to uncontrolled proliferation. Some cell lines originate from the cells that have been artificially immortalized by various methods.

The term “stem cell” and the related terms and expressions are used herein to refer to animal cells that are capable of dividing and renewing themselves for long periods, are unspecialized, and can give rise to specialized cell types. Stem cells are capable of dividing and renewing themselves for long periods. Unlike, for example, muscle cells, blood cells, or nerve cells—which do not normally replicate themselves—stem cells may replicate many times or proliferate. If the resulting cells continue to be unspecialized, like the parent stem cells, the cells are said to be capable of long-term self-renewal.

The term “self-renewal,” when used in reference to cells, describes their ability to divide and generate at least one daughter cell with the self-renewing characteristics of the parent cell, although one or more of other daughter cells may commit to a particular differentiation pathway. For example, a self-renewing hematopoietic stem cell can divide and form one daughter stem cell and another daughter cell committed to differentiation in the myeloid or lymphoid pathway. Non self-renewing cells can still undergo cell division to produce daughter cells, neither of which have the differentiation potential of the parent cell type, but instead generates differentiated daughter cells.

The terms “pluripotent,” “pluripotency” and the related terms and expressions refer to animal cells or cell populations with the ability to give rise to progeny that can undergo differentiation, under appropriate conditions, into cell types that collectively demonstrate characteristics associated with cell lineages from all of the three germ layers (endoderm, mesoderm, and ectoderm). For example, the expression “pluripotent stem cell characteristics” refers to characteristics of a cell or a cell population that distinguish pluripotent stem cells or their populations from other cells. The ability to give rise to progeny that can undergo differentiation, under the appropriate conditions, into cell types that collectively demonstrate characteristics associated with cell lineages from all of the three germ layers (endoderm, mesoderm, and ectoderm) is a pluripotent stem cell characteristic. Cell morphologies as well as expression or non-expression of certain combinations of molecular markers are also pluripotent stem cell characteristics. Pluripotent stem cells (PSCs) include embryonic stem cells (ESCs) and induced pluripotent stem cells (iPSCs). Embryonic stem cells (ESCs) are derived from embryos and, under appropriate conditions, they can remain undifferentiated (unspecialized) in culture. An embryonic stem cell line is a line of ESCs cultured under the conditions that allow proliferation without differentiation for months to years. Under other conditions, for example, if the cells are allowed to clump together to form embryoid bodies, they begin to differentiate spontaneously.

An “adult stem cell,” which can also be termed “somatic stem cell,” is a stem cell found, in an organism, among differentiated cells in a tissue or organ and can differentiate to yield some or all of the specialized cell times in the tissue or organ. Somatic stem cells can be grown in culture. When differentiating into specialized cells, they typically generate intermediate cells called “precursor” or “progenitor” cells. Somatic stem cells and progenitor cells can be described as “multipotent” or “oligopotent,” depending on their degree of potency. Some examples of somatic stem cells are: hematopoietic stem cells that give rise to all the types of blood cells (red blood cells, B lymphocytes, T lymphocytes, natural killer cells, neutrophils, basophils, eosinophils, monocytes and macrophages); mesenchymal stem cells that include bone marrow stromal stem cells and skeletal stem cells and can give rise to bone cells (osteoblasts and osteocytes), cartilage cells (chondrocytes), fat cells (adipocytes), and stromal cells that support blood formation; neural stem cells that can give rise to nerve cells (neurons), astrocytes and oligodendrocytes; epithelial stem cells in the lining of the digestive tract that can give rise to absorptive cells, goblet cells, Paneth cells, and enteroendocrine cells; skin stem cells that occur in the basal layer of the epidermis (and can give rise to keratinocytes) and at the base of hair follicles (and can give rise to both the hair follicle and to the epidermis). A tissue-specific progenitor cell is a cell devoid of self-renewal potential that is committed to differentiate into cells of a specific organ or tissue. Certain somatic stem cell types can differentiate into cell types seen in organs or tissues other than those expected from the somatic stem cell's origin. This phenomenon is called “transdifferentiation.”

The term “neural crest cells” (singular—“neural crest cell”) refers to specific cells at the border of the neural plate and the non-neural ectoderm that transiently exist during early embryo development. They can be considered multipotent stem cells. Neural crest cells give rise to most of the peripheral nervous system, including nociceptors, and to various non-neural cell types and tissues, such as smooth muscle cells, pigment cells in the skin, craniofacial bones, cartilage and connective tissue. Neural crest cells exist transiently during development, at a time point when the somatic body plan is not established yet. Therefore, they are generally not considered somatic stem cells.

The expression “induced pluripotent stem cell” (iPSC) refers to a pluripotent stem cell artificially derived from a non-pluripotent cell. For example, human iPSCs are artificially derived from a human non-pluripotent cell. iPSCs can be derived by introducing products of specific sets of pluripotency-associated genes, or “reprogramming factors,” into a given cell type and/or exposing non-pluripotent cells to particular conditions.

The term “non-pluripotent cells” refer to mammalian cells that are not pluripotent cells. Examples of such cells include differentiated cells, somatic stem cells, as well as progenitor cells. Some non-pluripotent cells maintain a degree of potency, some of the examples being somatic stem cells and progenitor cells.

“Cell potency” describes a cell's ability to differentiate into other cell types. A cell can be designated as a pluripotent cell, a multipotent cell (which can differentiate into several but not all cell types, for example, umbilical cord blood stem cells and mesenchymal stem cells) or an oligopotent cell (having the ability to differentiate into a few cell types, for example, lymphoid cells or vascular cells). Under current understanding, potency exists on a continuum. Thusly, the boundaries between the divisions of cells based on potency may be fluid and are not necessarily limiting.

The terms “progenitor cell” or “precursor cell,” as used herein, refers to the cells that can typically differentiate to form one or more kinds of cells. A “precursor cell” or “progenitor cell” can be any cell in a cell differentiation pathway that is capable of differentiating into a more mature cell. Progenitor cells can be primary cells obtained from an organism, cells proliferated in culture or cells derived from stem cells. Progenitor cells can be an early descendant or a pluripotent stem cell or a pluripotent cell itself. Progenitor cells can also be a partially differentiated multipotent cell or reversibly differentiated cell. The term “precursor cell population” refers to a group of cells capable of developing into a more mature or differentiated cell type. A precursor cell population can comprise cells that are pluripotent, cells that are stem cell lineage restricted (cells capable of developing into less than all lineages, or into, for example, only cells of neuronal lineage), and cells that are reversibly stem cell lineage restricted. Therefore, the term “progenitor cell” or “precursor cell” may be a “pluripotent cell” or “multipotent cell.”

“Differentiation” is the process by which a less specialized cell becomes a more specialized cell type. For example, early development of a multicellular animal is characterized by the rapid proliferation of embryonic cells, which then differentiate to produce the many specialized types of cells that make up the tissues and organs of the multicellular animal. As cells differentiate, their rate of proliferation usually decreases. Some types of differentiated cells never divide again, but many differentiated cells are able to resume proliferation as required to replace cells that have been lost as a result of injury or cell death. Some cells divide continuously throughout life to replace cells that have a high rate of turnover in adult multicellular animals. Examples of differentiated cells include, but are not limited to, cells from a tissue selected from bone marrow, skin, skeletal muscle, fat tissue and peripheral blood. Exemplary differentiated cell types include, but are not limited to, fibroblasts, hepatocytes, myoblasts, neurons, osteoblasts, osteoclasts, and lymphocytes.

The expression “modified cells” and the related terms and expressions encompass all cells that have been or are derived from the cells that have been artificially modified, by any methods, as compared to the original or cells from which they are derived. Modified cells can be produced from primary cells, secondary cells, stem cells, cultured cells and/or other modified cells. Modifications include, but are not limited to, genetic modification or engineering, in which case modified cells can be referred to as “genetically modified” or “genetically engineered.” Genetic modification can be accomplished by various methods that result in incorporation of foreign or heterologous nucleic acids into the cells being modified. Some examples of such methods are transduction by a virus or a viral vector, or transfection of isolated nucleic acids into cells through transient pores in the cell membrane. Other modifications include exposing the source cells to biological and non-biological molecules or factors or culture conditions. Some examples of modified cells are iPSCs, genetically modified cells, including those used for gene therapies, one example being gene-edited cells, such as those modified using CRISPR/Cas9, TALENs or ZFNs.

The term “vessel” refers to a container, dish, plate, flask, bottle, cell culture tube, a bioreactor and the like, which can be used to culture, maintain or grow a cell, group of cells, tissue or organ ex vivo or in vitro. Suitable vessels include, for example, multi-well plates, wells of multi-well plates, dishes, tubes, flasks, bottles and reactors.

The terms “stabilize” and the related terms and expressions used in reference to cells (for example, “stabilizing a cell”) refer to reduction of negative cell responses, such as cell death or senescence. For example, stem cells and other cells can die in response to cell passaging, dissociation, isolation, freezing and/or thawing. In other words, the above conditions can reduce cell viability. Embodiments of the compositions, methods and kits described therein can mitigate the reduction of cell viability and improve cell survival, which can be described as cell stabilization.

The term “spheroids,” “spheres” or “nocispheres” as used herein, and the related terms and expressions can be used in this document to refer to self-assembling, free-floating cell aggregates of undifferentiated and/or differentiating cells, which can be grown in suspension in low-attachment plates, spinner flasks or other vessels.

As used herein, “marker” refers to any molecule that can be observed or detected. For example, a marker can include, but is not limited to, a nucleic acid, such as a transcript of a specific gene, a polypeptide product of a gene, a non-gene product polypeptide, a glycoprotein, a carbohydrate, a glycolipid, a lipid, a lipoprotein or a small molecule (for example, molecules having a molecular weight of less than 10,000 AMU).

In the context of observable markers of cell development or differentiation, “expression” refers to the production of a gene product (which can be a nucleic acid, such as RNA, or a protein) as well as the level or amount of production of a gene product. Thus, determining the expression of a specific marker refers to detecting either the relative or absolute amount of the marker that is expressed or simply detecting the presence or absence of the marker. For most markers described herein, the symbols provided are those developed and/or recognized by HUGO Gene Nomenclature Committee of European Bioinformatics Institute.

The term “cryopreservation,” as well as related terms and expression, are used to refer to is a process or processes, as well as the results of such process or processes, by which cells, groups of cells or cell cultures re preserved by cooling to sub-zero temperatures.

Methods

Various methods (processes) are envisioned and included among the embodiments of the present invention. Among the methods according to embodiments of the present invention are methods of producing cells in culture of cells or cell cultures containing cells with at least some defined characteristics. Such methods can also be referred to “methods of cell production,” “method of cell culture production,” “methods of generating,” “methods of culturing,” “methods of differentiating,” “differentiation method,” “differentiation process” and by other related terms and phrases, which can be used interchangeably in reference to methods of producing cells or cell cultures. One example of such methods is a method of producing or generating multipotent cells, which are in turn capable of differentiating into cells exhibiting at least some characteristics of nociceptor cells. The multipotent cells produced by such methods exhibit at least some characteristics of neural crest cells, such as expression of SOX10. Accordingly, such multipotent cells can be referred to as “cells exhibiting at least some characteristics of neural crest cells,” “neural crest-like cells,” “cells resembling neural crest cells” and by other related terms and expressions. Cells exhibiting at least some characteristics of neural crest cells, along with the relevant characteristics, are discussed further in this document. One more example of a method according to embodiments of the present invention is a method of producing or generating cells exhibiting at least some characteristics of nociceptor cells, such as expression of one or more ion channels, receptors, etc. Cells exhibiting at least some characteristics of nociceptor cells produced according to the embodiments of the methods of the present invention can also be referred to as “nociceptor-like cells,” “cells resembling nociceptors” and by other related terms and expressions. Cells exhibiting at least some characteristics of nociceptor cells, along with the relevant characteristics, are discussed further in this document. The methods according to the above embodiments of the present invention and other embodiments related to cell production are conducted in culture and can be referred to as “methods of culturing” or “culturing.” Such methods typically proceed from, as starting materials or intermediate products, less differentiated cells possessing higher potency (such as pluripotent cells, progenitor cells, multipotent cells or oligopotent cells) and proceed to, as intermediate and/or end products, more differentiated cells with lower potency (such as multipotent cells, progenitor cells, oligopotent cells or differentiated cells). Accordingly, the methods can be referred to as “methods of differentiating cells,” even if the end product is or contains the cells that are not completely differentiated.

In some exemplary embodiments, the methods use pluripotent stem cells (PSCs) as a starting material. Such PSCs can be vertebrate PSCs, including mammalian PSCs or human PSCs (hPSCs). PSCs used in the methods according to the embodiments of the present invention can be isolated from natural sources or artificially derived PSCs, such as induced PSCs (iPSCs). Accordingly, the methods can be referred to as “methods of differentiating PSCs,” for example, methods of differentiating hPSCs, methods of differentiating PSCs, etc. PSCs can be maintained and expanded in culture, such as monolayer culture or appropriate 3D culture systems (for example, those using microcarriers) in a defined medium, such as, but not limited to, E8, E8 Flex, StemFlex, StemPro, mTeSR, mTeSR1, StemFit, Nutristem, L7 Medium or iPS-Brew. The above maintenance and/or expansion of PSCs can be conducted as a part of the methods according to the embodiments of the present invention, or outside of such methods. In other words, cell production methods according to the embodiments of the present invention are not limited by the steps or processes employed to provide PSCs used for further steps, unless such limitations are explicitly stated. For example, if PSCs are simply listed as a starting material or “provided” without further limitations, then the processes used to obtain, culture, expand or grow PSCs are not intended to be incorporated into the method. PSCs can be provided in the form of monolayer cultures exhibiting, for example, typical PSC morphology, which may include prominent nucleoli and/or high nuclear-to-cytoplasmic ratio, cell growth in colonies, and expression of pluripotency-associated markers such as, but not limited to, OCT3/4, NANOG, SSEA-4, TRA-1-60, TRA-1-81 and/or Alkaline Phosphatase. In another example, PSCs can be provided in the form of 3D cultures or attached to microcarriers.

Cell production methods according to the embodiments of the present invention can include a step of incubating vertebrate PSCs (which can be ESCs or iPSCs), such as human PSCs, in a defined medium. The PSCs being incubated can be in a form of an adherent monolayer culture, which can be provided at >90% confluence at the start of a cell production method according to embodiments of the present invention. Some non-limiting examples of defined media suitable for incubating PSCs are E6, DMEM/F12 and DMEM/KnockOut. The defined medium contains an effective amount or concentration of at least one compound capable of activating WNT signaling (WNT activator), such as CHIR98014 at 20 nM-20 μM or 100 nM-10 μM (in one example, 1 μM), or CHIR99021 at 20 nM-20 μM or 100 nM-10 μM (in one example, 1 μM), and an effective amount or concentration of at least one compound capable of inhibiting TGF-beta signaling, such as A83-01 at 20 nM-20 μM or 100 nM-10 μM (in one example, 2 μM), or SB431542 at 20 nM-40 μM or 100 nM-40 μM (in one example, 2 μM). It is to be understood that one or both the compound capable of activating WNT signaling and the compound capable of inhibiting TGF-beta signaling can be a small molecule or a peptide or protein molecule (either of which can be extracted from a natural source, chemically, biochemically or recombinantly produced; for example, recombinant proteins WNT3A, WNT5A and others can be used to activate WNT signalling). A period of time for incubating the monolayer cultures of PSCs is approximately 24 to approximately 144 hours, approximately 48 to approximately 112 hours, approximately 64 to approximately 80 hours, or approximately 72 hours (for example, 72 hours±7.2 hours). In some embodiments of the methods, additives activating or inhibiting bone morphogenic protein (BMP) protein pathways, such as Bone Morphogenic Protein 4 (BMP4), are not used in the medium in which the PSCs are incubated. In other words, the medium in which the PSCs are incubated is free or substantially free of exogenously supplemented additives activating or inhibiting bone morphogenic protein (BMP) protein pathways, such as Bone Morphogenic Protein 4 (BMP4), BMP2, small molecule inhibitors of the BMP pathway, such as Dorsomorphin or LDN193181, or others.

After the above-described incubation step, a monolayer culture of differentiating PSCs (since differentiation is ongoing and not complete, it is appropriate to refer to the cells at this point as differentiating PSCs) is dissociated, for example, by enzymatic dissociation. Enzymatic dissociation can be performed by removing the incubation medium from the plates, adding to the plates a buffer, such as PBS and an enzymatic dissociation reagent, such as Accutase, TrypLE or Trypsin available from Thermo Fisher Scientific, incubating the cells with the buffer and dissociation reagent under appropriate conditions, and harvesting the resulting single cells by centrifugation, sedimentation, filtering or other appropriate methods. The dissociated cells are transferred into similar or equivalent uncoated or ultra-low attachment cells or flasks using a so-called “1:1 transfer” procedure. For example, dissociated cells from a six-well plate can be transferred into a similar six-well ultra-low attachment plate. In another examples, cells from coated flasks, such as T75 or T175 flasks (Corning), can be transferred into the same number of uncoated or ultra-low attachment T75 or T175 flasks. After the transfer, the cells are cultured under conditions leading to formation of nocispheres, which are self-assembling, free-floating cell aggregates of differentiating cells, at least some of which are neural crest-like (as discussed elsewhere in this document) that produce nociceptor-like cells when subjected to the method steps described further in this document.

In one example, the culture conditions leading to formation of nocispheres include incubation in a defined medium, such as, but not limited to, E6, DMEM/F12 or Knockout DMEM, supplemented with at least the following: an effective amount or concentration of at least one compound capable of activating WNT signaling (WNT activator), such as CHIR98014 at 20 nM-20 μM or 100 nM-10 μM (in one example, 1 μM), or CHIR99021 at 20 nM-20 μM or 100 nM-10 μM (in one example, 1 μM); an effective amount or concentration of at least one compound capable of inhibiting TGF-beta signaling, such as A83-01 at 20 nM-20 μM or 100 nM-10 μM (in one example, 2 μM), or SB431542 at 20 nM-40 μM or 100 nM-40 μM (in one example, 2 μM); an effective amount or concentration of at least one compound that is a Notch-pathway inhibitor, for example, a gamma-secretase inhibitor DBZ at 20 nM-20 μM or 100 nM-10 μM (in one example, 1 μM), DAPT at 5 nM-50 μM or 100 nM-50 μM (in one example, 1 μM), LY411575 at 2 nM-20 μM or 100 nM-10 μM (in one example, 1 μM) or LY3039478 at 2 nM-20 μM or 100 nM-10 μM (in one example, 1 μM), and an effective amount or concentration of at least one compound that is a FGFR/VEGFR/MAP kinase pathway inhibitor, such as PD173074 2 nM-20 μM or 2 nM-5 μM (in one example, 25 nM), or SU5402 at 2 nM-20 μM or 2 nM-10 μM (in one example, 25 nM). It is to be understood that one or more of the compound capable of activating WNT signaling, the compound capable of inhibiting TGF-beta signaling, the compound that is a Notch-pathway inhibitor or the compound that is a FGFR/VEGFR/MAP kinase pathway inhibitor can be a small molecule or a peptide or protein molecule (either of which can be extracted from a natural source, chemically, biochemically or recombinantly produced; for example, recombinant proteins WNT3A, WNT5A and others can be used to activate WNT signalling), in any combination. In some embodiments, the defined medium can be further supplemented with PD0332991, which is a CDK4/6 inhibitor, used at a concentration of from approximately 2 nM to approximately 20 such as 1 or any other small molecule, protein or peptide (any of which can be chemically, biochemically or recombinantly produced) CDK 4/6 inhibitor. In some embodiments of the methods, compounds activating or inhibiting bone morphogenic protein (BMP) protein pathways, such as Bone Morphogenic Protein 4 (BMP4), or small molecule inhibitors of BMP protein pathways, such as Dorsomorphin or LDN193189, are not used (or absent) from the medium in which the PSCs are cultured in order to form nocispheres. During nocisphere formation, the medium containing the additives can be changed approximately every 12-36 hours, for example, approximately every 24 hours. The culturing leading to nocisphere formation is conducted for approximately 168 to approximately 432 hours, for example, approximately 168 hours, approximately 192 hours, approximately 216 hours, approximately 240 hours, approximately 264 hours, approximately 288 hours, approximately 312 hours, approximately 336 hours, approximately 360 hours, approximately 384 hours, approximately 408 hours or approximately 432 hours. In one example, the culturing is conducted for 258-260 hours. It is to be understood that the above conditions for nocisphere formation are exemplary, and other conditions may be used to generate and maintain viable free-floating nocispheres. The nocispheres can contain varying proportions of neural crest-like cells (which may be characterized by expression of SOX10) and nociceptor-like cells (which may be characterized by expression of BRN3A). In one example, a nocisphere can contain approximately 50%, approximately 60%, approximately 70%, approximately 80%, approximately 90% or over 90% of neural crest-like cells. In another example, a nocisphere can contain approximately 50%, approximately 40%, approximately 30%, approximately 20%, approximately 10% or less than 10% of nociceptor-like cells.

After the nocisphere formation step, the nocispheres are dissociated, for example, by enzymatic dissociation. The dissociated nocisphere cells are then cultured in a monolayer culture. The nocispheres can be cryopreserved prior to or after the dissociation and prior to culturing in a monolayer culture. The culturing in the monolayer culture can be conducted for periods of time encompassing days to months. For example, the cells can be cultured for at least approximately 24 hours (1 day), approximately 24 hours (1 day), at least approximately 48 hours (2 days), approximately 48 hours (2 days), at least approximately 72 hours (3 days), approximately 72 hours (3 days), at least approximately 96 hours (4 days), approximately 96 hours (4 days), at least approximately 120 hours (5 days), 120 hours (5 days), at least approximately 144 hours (6 days), 144 hours (6 days), at least approximately 168 hours (7 days), 168 hours (7 days), at least approximately 192 hours (8 days), 192 hours (8 days), at least approximately 216 hours (9 days), 216 hours (9 days), at least approximately 240 hours (10 days), 240 hours (10 days), at least approximately 264 hours (11 days), 264 hours (11 days), at least approximately 288 hours (12 days), 288 hours (12 days), at least approximately 312 hours (13 days), 312 hours (13 days), at least approximately 336 hours (14 days), 336 hours (14 days), etc. The culturing in the monolayer culture can end when the cultured cells are harvested. The cultured cells can be cryopreserved, used in various applications, some of which are described elsewhere in this document, or used for further culturing. In one example, the culturing is conducted for approximately 168-336 hours. For example, the dissociated nocisphere cells can be cultured in an appropriate medium, such as DMEM/F12, supplemented with a suitable supplement or combination of supplements. In one example, a combination of N2 supplement and B27 supplement can be used. In another example, a combination of a combination of N2 supplement and B27 supplement (without vitamin A) can be used. In one more example, a combination of N2 supplement, B27 supplement (without vitamin A) can be used, BDNF, GDNF, NGF and NT-3 can be used. In yet one more example, any of the above supplement combinations can be further used in combination if PD0332991, which a CDK4/6 inhibitor, used at a concentration of from approximately 2 nM to approximately 20 μM, such as 1 μM, or any other small molecule, protein or peptide (any of which can be chemically, biochemically or recombinantly produced) CDK 4/6 inhibitor. In some embodiments, the supplements include one or more of: an effective amount or concentration of at least one compound that is a Notch-pathway inhibitor, for example, a gamma-secretase inhibitor DBZ at 20 nM-20 μM or 100 nM-10 μM (in one example, 1 μM), DAPT at 5 nM-50 μM or 100 nM-50 μM (in one example, 1 μM), LY411575 at 2 nM-20 μM or 100 nM-10 μM (in one example, 1 μM) or LY3039478 at 2 nM-20 μM or 100 nM-10 μM (in one example, 1 μM), and an effective amount or concentration of at least one compound that is a FGFR/VEGFR/MAP kinase pathway inhibitor, such as PD173074 2 nM-20 μM or 2 nM-5 μM (in one example, 25 nM), or SU5402 at 2 nM-20 μM or 2 nM-10 μM (in one example, 25 nM), and an effective amount of concentration of at least one compound that is a CDK4/6 inhibitor, such as if PD0332991 used at a concentration of from approximately 2 nM to approximately 20 μM, such as 1 μM. It is to be understood that one of more of the compound that is a Notch-pathway inhibitor, the compound that is a FGFR/VEGFR/MAP kinase pathway inhibitor or the compound that is a CDK4/6 inhibitor can be a small molecule or a peptide or protein molecule (either of which can be extracted from a natural source, chemically, biochemically or recombinantly produced), in any combination. Culturing of dissociated nocisphere cells in a monolayer culture generates a culture containing cells neural crest-like cells exhibiting at least some characteristics, such as SOX10 expression, of natural neural crest cells, and nociceptor-like cells exhibiting at least some characteristics of naturally occurring nociceptors, such as BRN3A expression. In other words, SOX10 positive neural crest-like cells and BRN3A positive nociceptor-like cells can both be generated and co-exist in a culture of dissociated nocisphere cells. For example, at the start of culturing dissociated nocisphere cells, the culture can contain a mixture of SOX10- and BRN3A-expressing cells in various proportions, which can change over the course of culturing. In one example, a culture of nocisphere dissociated cells at the start of the culture can contain approximately 50%, approximately 60%, approximately 70%, approximately 80%, approximately 90% or over 90% of SOX-10 expressing cells In another example, a culture of nocisphere dissociated cells at the start of the culture can contain can contain approximately 50%, approximately 40%, approximately 30%, approximately 20%, approximately 10% or less than 10% of BRN3A-expressing cells. The above proportions can change over the course of the culture changes as differentiation of neural crest-like cells into nociceptor-like cells occurs. As an illustration, at the start of the culture, a culture of nocisphere dissociated cells can contain approximately 60% of SOX10-expressing cells and approximately 40% of BRN3A expressing cells, while after 14 days, the same culture can contain approximately 80% of SOX10-expressing cells and approximately of 20% BRN3A expressing cells.

The cells exhibiting at least some characteristics of neural crest cells (neural crest-like cells), which are capable of differentiating into the cells exhibiting the at least some characteristics of the nociceptor cells (nociceptor-like cells), as well as the mixture of neural crest-like cells and nociceptor-like cells (for example, produced at nocisphere formation step or by culturing of dissociated nocisphere cells) can be the end product of some, but not all, of the methods according to the embodiments of the present invention. Neural crest-like cells or a mixture of neural crest-like cells and nociceptor-like cells can be an intermediate of some of the methods according to embodiments of the present invention, and can also be a starting material according to some other methods according to the embodiments of the present invention. Neural crest-like cells or mixtures of neural crest-like cells and nociceptor-like cells can be prepared for cryopreservation and cryopreserved. The method steps related to cryopreservation can be incorporated into the methods of cell generation according to the embodiments of the present invention. Some of the methods and compositions relevant to cryopreservation are described further in this application in the section “Cryopreservation,” although is to be understood that the description provided that section is not limiting, and that other compositions and methods can be employed for cryopreservation. In some embodiments of the methods according to the present invention, neural crest-like cells or mixtures of neural crest-like cells and nociceptor-like cells can be cultured in order to increase their numbers. For example, cryopreserved cells can be thawed and cultured in mitogens such as fibroblast growth factor 2 (FGF-2) and epidermal growth factor (EGF). In one example, for further differentiation and maturation, neural crest-like cells or a mixture of neural crest-like cells and nociceptor-like cells can be cultured on coated plates (plates coated, for example, with Geltrex, Matrigel or laminin) using DMEM/F12 supplemented with N2 supplement, B27 supplement (without Vitamin A), BDNF, GDNF, NGF, NT-3 and 1 μM PD0332991.

The cells exhibiting at least some characteristics of neural crest cells are capable of differentiating, under appropriate conditions, into cells exhibiting at least some characteristics of nociceptor cells. Methods of producing, in culture, cells exhibiting at least some characteristics of nociceptor cells (nociceptor-like cells) are included among the embodiments of the present invention. Neural crest-like cells, or cultures including such cells can be a starting material or intermediate of such methods. In one example, cells including the cells exhibiting at least some characteristics of neural crest cells (neural crest-like cells) are cultured under conditions inducing their differentiation into cells exhibiting the at least some characteristics of the nociceptor cells (nociceptor-like cells). Accordingly, an embodiment of a method based on the above example can include a step of growing neural crest-like cells in culture in a suitable medium for a suitable period of time. The above step of growing can also be referred to as “maturation,” “differentiation,” “incubation” or by other related terms and expressions, which do not imply additional limitations unless those are explicitly stated. The incubation can be conducted in monolayer cell culture, although other types of culture can also be used, such as 3D organoids or bioprinted tissues to generate dorsal root ganglia in order to mimic in vivo anatomy. Methods according to embodiments of the present invention can include a step of establishing a culture prior to differentiation, such as plating neural crest-like cells onto plates for incubation. The plates used for monolayer cell culture can be of any appropriate type, some examples being coated cell culture plates, such as, but not limited to, laminin-coated plates (BioCoat from Corning) or CellBIND (Corning). The medium used for incubation can be of any appropriate type, some non-limiting examples being DMEM/F12, Neurobasal (Thermo Fisher Scientific) or BrainPhys (Stem Cell Technologies). The medium can be supplemented with effective amounts or concentrations of one or more supplements useful for supporting differentiation cells in culture into cells exhibiting characteristics of neural cells. Some examples of such supplements are: commercially available supplements, such as N2 supplement (dilute 100× solution provided by Thermo Fisher Scientific) or B27 supplement without vitamin A (dilute 50× solution provided by Thermo Fisher Scientific); biological molecules, such as growth factors, for example Brain-Derived Neurotrophic Factor (BDNF), Glial-Derived Neurotrophic Factor (GDNF), Nerve Growth Factor (NGF), Neurotrophin-3 (NT-3), Midkine or Pleiotrophin; or small molecules, such as Forskolin and cyclic adenosine monophosphate. Culturing can be performed under certain conditions, such as 5% low oxygen conditions to mimic in vivo oxygen content.

It is to be understood that various events or scenarios may occur in the culture of neural crest-like cells. For example, neural crest-like cells can differentiate into nociceptor-like cells or other types of cells. Neural crest-like cells can also continue proliferating without further differentiation. Such events can be occurring simultaneously, with some of the neural crest-like cells in the culture differentiating, and some other neural crest-like cells continuing to proliferate without further differentiation. Various additives can be used in order to promote some of the events and inhibit other events, as desired. For example, when neural crest-like cells are cultured in order to increase their numbers, such as after cryopreservation, it is desirable to inhibit differentiation and/or promote proliferation of neural crest-like cells. In another example, when neural crest-like cells are cultured in order to differentiate them into nociceptor-like cells or other cell types, it is desirable to promote differentiation and inhibit proliferation. In some embodiments, in order to promote differentiation of neural crest-like cells, various compounds can be added to the medium at various points during the differentiation step. Some examples of such inhibitors, which can be used in various combinations, are: a CDK4/6 inhibitor, such as PD0332991 at 2 nM-20 μM or 100 nM-10 μM (in one example, 1 μM) can be added to the medium to promote differentiation; FGF/VEGF/MAP kinase pathway inhibitor, such as PD 173974 at 2 nM-20 μM or 2 nM-5 μM (in one example, 25 nM), or SU5402 at 2 nM-20 μM or 2 nM-10 μM (in one example, 500 nM); an inhibitor of Notch pathway, such as a gamma-secretase inhibitor DBZ at 20 nM-20 μM or 100 nM-10 μM (in one example, 1 μM), DAPT at 5 nM-50 μM or 100 nM-50 μM (in one example, 1 μM), LY411575 at 2 nM-20 μM or 100 nM-10 μM (in one example, 1 μM) or LY3039478 at 2 nM-20 μM or 100 nM-10 μM (in one example, 1 μM). In some embodiments, in order to achieve preferential proliferation of neural crest-like cells (over their differentiation), the medium can be supplemented with mitogens such as fibroblast growth factor 2 (FGF-2) and epidermal growth factor (EGF).

The efficiency of the described method steps can be adjusted by modifying certain parameters, which include but are not limited to, cell growth conditions, additive concentrations and the timing of the steps. The method steps described herein can result in about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or greater than about 95% conversion of less differentiated cells possessing higher potency (such as pluripotent cells, progenitor cells, multipotent cells or oligopotent cells) to more differentiated cells with lower potency (such as multipotent cells, progenitor cells, oligopotent cells or differentiated cells). Examples of the conversion steps that can characterized by the above degrees of efficiency are conversion of PSC to neural crest-like cells, conversion of neural crest-like cells to nociceptor-like cells, or conversion of PSCs to nociceptor-like cells. In one example, starting with 1 million PSCs, at day 14 it is possible to generate a mixture of 2.0-2.5 million SOX10-positive neural crest-like cells and BRN3A-positive nociceptor-like cells. At day 28, total cell numbers can remain constant but the fraction of differentiated nociceptor-like cells increases, if proliferation is inhibited and maturation facilitated.

### Automation

Automated methods of cell culture are included among the embodiments of the present invention. Also included among the embodiments of the present invention are systems for performing or partially performing embodiments of the automated methods of the present invention. The systems according to the embodiments of the present invention may include various stations and/or components, some examples of which are described below. As used herein, the term “station” is broadly defined and includes any suitable apparatus or assemblies, conglomerations or collections of apparatuses or components suitable for carrying out a method according to the embodiments of the present invention. The stations need not be integrally connected or situated with respect to each other in any particular way. Systems according to the embodiments of the present invention may include any suitable arrangements of the stations with respect to each other. For example, the stations need not even be in the same room. But in some embodiments, the stations are connected to each other in an integral unit.

Automated cell culture methods and system for performing various methods according to embodiments of the present invention may be used to optimize conditions of various method steps and/or and to scale up production of cells produced by the methods, such us neural crest-like cells and/or nociceptor-like cells. In general, automated methods and systems according to the embodiments of the present invention minimize human intervention needed during cell culture procedures such as feeding, passing or harvesting of cells. In addition to freeing up laboratory personnel, the disclosed automated methods and systems allow for these techniques to be carried out in a reliable and reproducible manner. For example, a system for performing various methods according to embodiments of the present invention may include a station for robotic or automated cell culture, one example of which is CompacT SelecT® (Sartorius, Wilmington, Del.) system. An automated cell culture system can grow, expand, and differentiate cells by performing methods according to the embodiments of the present invention. An automated cell culture system may be able to perform one or more steps required for cryopreservation of cells. An automated cell culture system can perform one or more cell culture processes, such as, but not limited to, seeding cell culture flasks or plates, maintaining cell cultures, for example, in cell culture flasks or plates, harvesting cells, pooling cells from harvesting flasks or plates, diluting cells for sub-culturing an plating, conducting cell counts, conducting cell viability assays, etc. An automated cell culture systems can include various stations, such as, but not limited to: a station for incubating cells, which is exemplified by an automated flask incubator maintaining a controlled environment (including controlled temperature, controlled gas composition and/or aseptic environment maintenance); a station for handling of flasks and other cell culture instruments, such as pipettes, which can be exemplified by a robotic arm or other type of robotic handler); a station for reagent dispensing, such as a robotic low volume dispenser; etc.

An automated cell culture system can include various computer components. An automated cell culture system embodiment, or parts of the system, may be controlled by a computer. For example, an automated cell culture system may include a computer-based station for generating reports. An automated cell culture system may include a computer-based station or components for data analysis. An automated cell culture system may include a computer, a processor, electronic memory, software instructions etc. An automated cell culture system may include software instructions for one or more of: system operation, workflow optimization, auditing and/or tracking of cell culture flasks or plates, etc. For example, an automated cell culture system may include an application software program to run programmed protocols on the robotic liquid handling system. The software program may run on an external device (for example, a portable computer, such as a tablet computer or a smartphone) which is in communication with a controller built into the robotic liquid handling system; the software program in some embodiments may coordinate control of the robotic liquid handling system and, when present, the external robotic system as well, to implement at least some steps of the methods according to the embodiments of the present invention. The software program may be programmed to alert users, for example, using sound, light, vibration, email alerts, text alerts, when intervention is needed, either due to a fault/error or due to a procedure being completed.

### Computer-Based Calculations and Tools

The methods described in this document can involve computer-based calculations and tools. Tools can be advantageously provided in the form of computer programs that are executable by a general-purpose computer system (which can be called “host computer”) of conventional design. The host computer may be configured with many different hardware components and can be made in many dimensions and styles (for example, desktop PC, laptop, tablet PC, handheld computer, server, workstation, mainframe). Standard components, such as monitors, keyboards, disk drives, CD and/or DVD drives, and the like, may be included. Where the host computer is attached to a network, the connections may be provided via any suitable transport media (e.g., wired, optical, and/or wireless media) and any suitable communication protocol (e.g., TCP/IP); the host computer may include suitable networking hardware (e.g., modem, Ethernet card, WiFi card). The host computer may implement any of a variety of operating systems, including UNIX, Linux, Microsoft Windows, MacOS, or any other operating system.

Computer code for implementing aspects of the present invention may be written in a variety of languages, including PERL, C, C++, Java, JavaScript, VB Script, AWK, or any other scripting or programming language that can be executed on the host computer or that can be compiled to execute on the host computer. Code may also be written or distributed in low level languages such as assembler languages or machine languages.

The host computer system advantageously provides an interface via which the user controls operation of the tools. In the examples described herein, software tools are implemented as scripts (for example, using PERL), execution of which can be initiated by a user from a standard command line interface of an operating system such as Linux or UNIX. Commands can be adapted to the operating system as appropriate. In other embodiments, a graphical user interface may be provided, allowing the user to control operations using a pointing device. Thus, the present invention is not limited to any particular user interface.

Scripts or programs incorporating various features of the present invention may be encoded on various computer readable media for storage and/or transmission. Examples of suitable media include magnetic disk or tape, optical storage media such as compact disk (CD) or DVD (digital versatile disk), flash memory, and carrier signals adapted for transmission via wired, optical, and/or wireless networks conforming to a variety of protocols, including the Internet.

### Additives

Various additives can be used in the methods of cell production according to the embodiments of the present invention and in the related compositions and kits. Some additives and/or additive components are discussed below for clarity. It is understood that other additive and/or additive components may be used, even if they are not discussed below. In the context of the embodiments of the present invention, each of the components separately or a combination of components, can be referred to as “additive,” “supplement,” “active agent” or by other related terms, in singular or plural. Various formulations of the additives are envisioned. For example, additives can be formulated to contain amounts of one or more active agents sufficient to provide effective concentrations or effective amounts of the respective active agent or agents upon addition to culture media. In the context of the embodiment of the present invention, effective concentrations or effective amounts are those concentrations or amounts, respectively, of the one or more active agents that elicit desired effects on the cells exposed to the compositions, such as, but not limited to, improved survival (viability), cell stabilization, improved growth, reduced cell death, reduced senescence, improved growth, improved differentiation, etc. Additives are typically formulated so that they can be readily incorporated into culture media. For example, culture media additives can be provided in powdered form, as a tablet or as a capsule readily dissolvable in aqueous culture media. In another examples, additives can be provided as concentrated solutions or as suspensions to be added to culture media.

N-2 supplement is a chemically-defined, serum-free supplement based on Bottenstein, J. E. Cell Culture in the Neurosciences, Bottenstein, J. E. and Harvey, A. L., editors, p. 3-43, Plenum Press: New York and London (1985).

B-27 Supplement is an optimized serum-free supplement described, for example, in Brewer et al. Journal of Neuroscience Research 35:567-76, 1993.

Brain-Derived Neurotrophic Factor (BDNF) is a neurotrophic factor known to be capable of signaling through a high affinity cell surface receptor GP145/TrkB. Human BDNF is expressed as the C-terminal portion of a 247 amino acid polypeptide precursor, which also contains a signal sequence of 18 amino acid residue and a propeptide of 110 amino acid residues. When used as an additive, BDNF can be provided as recombinant human BDNF, produced, for example, as a 27.0 kDa homodimer of two 119 amino acid subunits linked by strong non-covalent interactions. An effective concentration of BDNF can be 1-100 ng/ml.

Glial-Derived Neurotrophic Factor (GDNF) is a member of the cysteine-knot superfamily of growth factors and is a glycosylated disulfide-linked homodimeric protein having a molecular weight of about 15 kDa. GDNF is known to signal through a multicomponent receptor system, composed of a RET and one of the four GFRα (α1-α4) receptor. When used as an additive, GDNF can be provided as recombinant human GDNF. An effective concentration of GDNF can be 1-100 ng/ml.

Nerve Growth Factor (NGF), also known as NFG-β is a well-characterized neurotrophic protein that plays a critical role in the development of sympathetic and some sensory neurons in the peripheral nervous system. When used as an additive, GDNF can be provided as recombinant human NGF. An effective concentration of NGF can be 1-100 ng/ml.

Neurotrophin-3 (NT-3) is a member of the neurotrophin family. The NT-3 cDNA encodes a 257 amino acid residue precursor protein with a signal peptide and a proprotein that are cleaved to yield the 119 amino acid residue of mature NT-3. Biologically active NT-3 is thought to be a non-covalently linked homodimer. NT-3 has identical amino acid sequence in human, mouse, and pig with full cross-reactivities. NT-3 is important in development and maintenance of neuronal population. An effective concentration of NT-3 can be 1-100 ng/ml

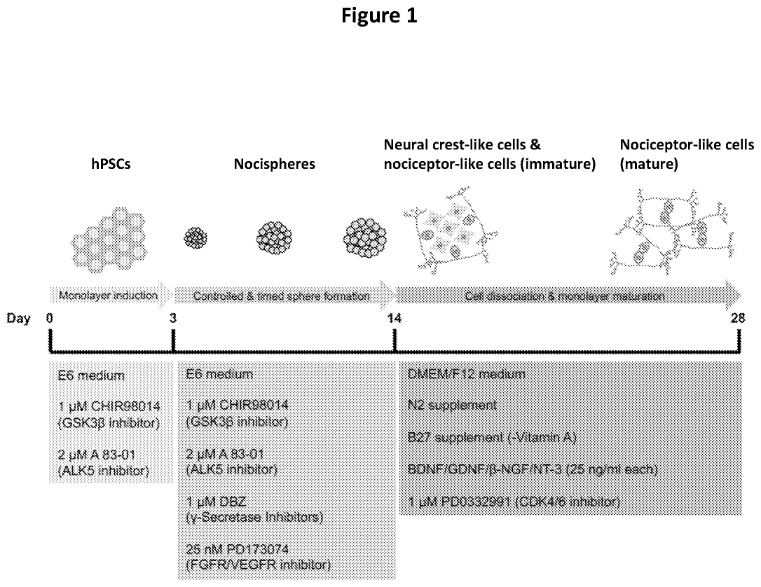

The term “Chroman 1” refers to (3S)-N-{2-[2-(Dimethylamino)ethoxy]-4-(1H-pyrazol-4-yl)phenyl}-6-methoxy-3,4-dihydro-2H-1-benzopyran-3-carboxamide, with the structure shown in FIG. 1. Chroman-related compounds or derivatives are structurally-related compounds (Chroman moiety-containing ROCK inhibitors), some of which are described in Chen et al. “Chroman-3-amides as potent Rho kinase inhibitors” Bioorganic and Medicinal Chemistry Letters 18:6406-6409 (2008) and LoGrasso et al. “Rho Kinase (ROCK) Inhibitors and Their Application to Inflammatory Disorders” Current Topics in Medicinal Chemistry 9:704-723 (2009). Chroman 1, its derivatives or related compounds can be supplied as a salt or in solution. An effective concentration of Chroman 1 (or its active derivative or a related compound) can be about 4 nM to about 80 μM, about 10 nM to about 20 μM, about 20 nM to about 10 μM or about 30 nm to about 500 nM, such as about 4 nm, 5 nM, 30 nM, 55 nM, 80 nM, 105 nM, 130 nM, 155 nM, 180 nM, 205 nM, 230 nM, 255 nM, 280 nM, 305 nM, 330 nM, 355 nM, 380 nM, 405 nM, 430 nM, 455 nM, 480 nM, 500 nM. 525 nM, 550 nM, 575 nM, 600 nM, 625 nM, 650 nM, 675 nM, 700 nM, 725 nM, 750 nM, 775 nM, 800 nM, 825 nM, 850 nM, 875 nM, 900 nM, 925 nM, 950 nM, 975 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM or 40 μM.

The term “Emricasan” refers to 3-(2-(2-tert-butylphenylaminooxalyl) aminopropionylamino)-4-oxo-5-(2,3,5,6-tetrafluorophenoxy)pentanoic acid, with the structure shown in FIG. 1. Emricasan-related compounds or derivatives are structurally-related compounds (such as Q-VD-OPh hydrate), some of which are described in Linton et al. “First-in-Class Pan Caspase Inhibitor Developed for the Treatment of Liver Disease” J. Med. Chem. 48:6779-6782, (2005). Emricasan, its derivatives or related compounds can be supplied as a salt or in solution. An effective concentration of Emricasan (or its active derivative or a related compound) can be about 5 nM to about 100 μM, about 200 nM to about 30 μM, about 300 nM to about 20 μM, for example, about 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 10.5 μM, 11 μM, 11.5 μM, 12 μM, 12.5 μM, 13 μM, 13.5 μM, 14 μM, 14.5 μM, 15 μM, 15.5 μM, 16 μM, 16.5 μM, 17 μM, 17.5 μM, 18 μM, 18.5 μM, 19 μM, 19.5 μM or 20 μM.

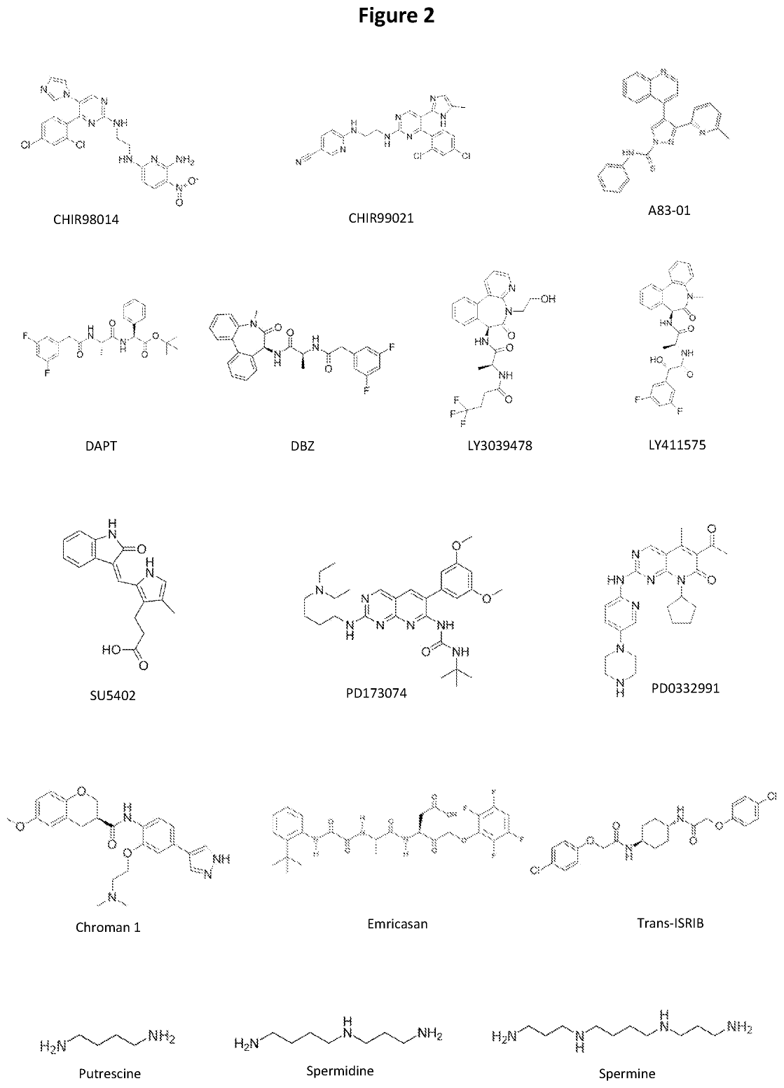

The term “trans-ISRIB,” which can be used interchangeably with the terms “ISRIB” or “ISRIB (trans-isomer)” refers to N,N′-((1r,4r)-cyclohexane-1,4-diyl)bis(2-(4-chlorophenoxy)acetamide) with the structure shown in FIG. 2. As described in Sidrauski et al. “Pharmacological brake-release of mRNA translation enhances cognitive memory” eLIFE 2:e00498 (2013), trans-ISRIB is 100-fold more potent (IC₅₀=5 nM) than cis-ISRIB (IC₅₀=600 nM) suggesting a stereospecific interaction with the cellular target. Trans-ISRIB can be supplied as a salt or in solution. An effective concentration of trans-ISRIB can be about 5 nM to about 50 μM, about 100 nM to about 6.25 μM, or about 200 nM to about 6.25 μM, for example, about 50 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 5.25 μM, 5.5 μM, 5.75 μM, 6 μM or 6.25 μM.

The term “polyamines,” as used herein, refers to one or more of the polycations putrescine, spermidine and spermine, which are known to interact with negatively charged macromolecules, such as DNA, RNA and proteins. An effective concentration of spermine can be about 0.5 μM to 1 mM, for example, about 0.5 μM, 20.5 μM, 40.5 μM, 60.5 μM, 80.5 μM, 100.5 μM, 120.5 μM, 140.5 μM, 160.5 μM, 180.5 μM, 200.5 μM, 220.5 μM, 240.5 μM, 260.5 μM, 280.5 μM, 300.5 μM, 320.5 μM, 340.5 μM, 360.5 μM, 380.5 μM, 400.5 μM, 420.5 μM, 440.5 μM, 460.5 μM, 480.5 μM, 500.5 μM, 520.5 μM, 540.5 μM, 560.5 μM, 580.5 μM, 600.5 μM, 620.5 μM, 640.5 μM, 660.5 μM, 680.5 μM, 700.5 μM, 720.5 μM, 740.5 μM, 760.5 μM, 780.5 μM, 800.5 μM, 820.5 μM, 840.5 μM, 860.5 μM, 880.5 μM, 900.5 μM, 920.5 μM, 940.5 μM, 960.5 μM, 980.5 μM or 1 mM. An effective concentration of spermidine can be about 0.5 μM to 1 mM, for example, approximately 0.5 μM, 20.5 μM, 40.5 μM, 60.5 μM, 80.5 μM, 100.5 μM, 120.5 μM, 140.5 μM, 160.5 μM, 180.5 μM, 200.5 μM, 220.5 μM, 240.5 μM, 260.5 μM, 280.5 μM, 300.5 μM, 320.5 μM, 340.5 μM, 360.5 μM, 380.5 μM, 400.5 μM, 420.5 μM, 440.5 μM, 460.5 μM, 480.5 μM, 500.5 μM, 520.5 μM, 540.5 μM, 560.5 μM, 580.5 μM, 600.5 μM, 620.5 μM, 640.5 μM, 660.5 μM, 680.5 μM, 700.5 μM, 720.5 μM, 740.5 μM, 760.5 μM, 780.5 μM, 800.5 μM, 820.5 μM, 840.5 μM, 860.5 μM, 880.5 μM, 900.5 μM, 920.5 μM, 940.5 μM, 960.5 μM, 980.5 μM or 1 mM. An effective concentration of putrescine can be about 0.1 μM to 2 mM.

The term “CHIR98014” refers to N⁶-[2-[[4-(2,4-Dichlorophenyl)-5-(1H-imidazol-1-yl)-2-pyrimidinyl]amino]ethyl]-3-nitro-2,6-pyridinediamine with the structure shown in FIG. 2. An effective concentration of CHIR98014 can be about 20 nM to 20 μM.

The term “CHIR99021” refers to 6-[[2-[[4-(2,4-Dichlorophenyl)-5-(5-methyl-1H-imidazol-2-yl)-2-pyrimidinyl]amino]ethyl]amino]-3-pyridinecarbonitrile with the structure shown in FIG. 2. An effective concentration of CHIR98014 can be about 20 nM to 20 μM.

The term “A83-01” refers to 3-(6-Methyl-2-pyridinyl)-N-phenyl-4-(4-quinolinyl)-1H-pyrazole-1-carbothioamide with the structure shown in FIG. 2. An effective concentration of A83-01 can be about 20 nM to 20 μM.

The term DBZ refers to N-[(1S)-2-[[(7S)-6,7-Dihydro-5-methyl-6-oxo-5H-dibenz[b,d]azepin-7-yl]amino]-1-methyl-2-oxoethyl]-3,5-difluorobenzeneacetamide, also known as dibenzazepine, with the structure shown in FIG. 2. An effective concentration of DBZ can be about 2 nM to 20 μM.

The term DAPT refers to (2S)-N-[(3,5-Difluorophenyl)acetyl]-L-alanyl-2-phenyl]glycine 1,1-dimethylethyl ester, with the structure shown in FIG. 2. An effective concentration of DAPT can be about 5 nM to 50 μM.

The term LY411575 refers to (S)-(+)-α-Amino-4-carboxy-2-methylbenzeneacetic acid, with the structure shown in FIG. 2. An effective concentration of LY411575 can be about 2 nM to 20 μM.

The term LY3039478 refers to 4,4,4-trifluoro-N-[(2S)-1-[[(7S)-5-(2-hydroxyethyl)-6-oxo-7H-pyrido[2,3-d][3]benzazepin-7-yl]amino]-1-oxopropan-2-yl]butanamide, with the structure shown in FIG. 2. An effective concentration of LY3039478 can be about 2 nM to 20 μM.

The term PD173074 refers to N-[2-[[4-(Diethylamino)butyl]amino-6-(3,5-dimethoxyphenyl)pyrido[2,3-d]pyrimidin-7-yl]-N′-(1,1-dimethylethyl)urea with the structure shown in FIG. 2. An effective concentration of PD173074 can be about 2 nM to 20 μM.

The term SU5402 refers to 2-[(1,2-Dihydro-2-oxo-3H-indol-3-ylidene)methyl]-4-methyl-1H-pyrrole-3-propanoic acid, with the structure shown in FIG. 2. An effective concentration of SU5402 can be about 2 nM to 20 μM.

The term PD0332991 refers to 6-acetyl-8-cyclopentyl-5-methyl-2-[[5-(1-piperazinyl)-2-pyridinyl]amino]pyrido[2,3-d]pyrimidin-7(8H)-one isethionate salt, also known as PD0332991 isethionate or Palbociclib, with the structure shown in FIG. 2. An effective concentration of PD0332991 can be about 2 nM to 20 μM.

### Cells, Compositions and Kits

Some embodiments of the methods of cell production described in this document involve, as a starting material or an intermediate, pluripotent or precursor cells or population of pluripotent or precursor cells or that are capable of selectively (and sometimes reversibly) developing into specified cellular lineages when cultured under appropriate conditions. As used herein, the term “population” refers to cell culture of more than one cell having the same identifying characteristics. The term “cell lineage” refers to all of the stages of the development of a cell type, from the earliest precursor cell to a completely mature cell (a specialized cell). One example of a precursor cell population that can be involved in the methods of cell production described in this document is a culture of pluripotent stem cells (PSCs), which may be a culture embryonic stem cells (ESCs) and induced pluripotent stem cells (iPSCs). Some embodiments of the methods of cell production described in this document involve human PSCs (hPSCs) or their populations as a starting material for deriving neural crest-like cells and nociceptor-like cells. It is to be understood that embodiments of the methods of cell production described in this document can involve modified PSCs, including hPSCs. Some examples of PSCs that can be used in the methods according to the embodiments of the present invention are various ESCs (e.g. WA01, WA09, WA14 from WiCell) and iPSC lines (LiPSC-GR1.1, NCRM-1, NCRM-2, NCRM-5, all available from National Institutes of Health (USA).

Another example of a precursor cell population that can be involved in the method of cell production described in this document is a population of neural crest-like cells, which can be produced from PSCs according to some embodiments of the methods described in this document. Neural crest-like cells, as discussed in this document, are cells exhibiting at least some properties of neural crest cells occurring during vertebrate embryonic development. During vertebrate embryonic development, the neural crest cells originate at the dorsalmost region of the neural tube. The neural crest cells migrate extensively to generate a large number of differentiated cell types, including the neurons and glial cells of the sensory, sympathetic, and parasympathetic nervous systems, the epinephrine-producing (medulla) cells of the adrenal gland, the pigment-containing cells of the epidermis and many of the skeletal and connective tissue components of the head. As currently understood, the fate of the neural crest cells depends, to a large degree, on where they migrate to and settle. A single neural crest cell can differentiate into any of several different cell types, depending on its location within the embryo. Thus, naturally occurring neural crest cells, as a population, are considered pluripotent or multipotent, although it is not currently clear if individual naturally occurring neural crest cells are pluripotent after leaving the neural crest or are already confined to certain lineages. Naturally occurring neural crest cells are characterized by expression of the so-called “specifiers,” a collection of genes including Slug/Snail, FoxD3, Sox10, Sox9, AP-2 and c-Myc. As currently understood, neural crest specifiers turn on the expression of effector genes, which control migration and multipotency. Effector genes include Rho GTPases, cadherins, Mitf, P0, Cx32, Trp and cKit. Neural crest-like cells according to some embodiments of the present invention express at least one marker of naturally occurring neural crest cells—SOX10. Neural crest-like cells according to some embodiments of the present invention can express one or more other markers of naturally occurring neural crest cells, such as SOX10, PAX3, NEUROG1, TFAP2A, TFAP2B. Neural crest-like cells possess the ability to differentiate, under appropriate culture conditions, into nociceptor-like cells. Compositions and kits including neural crest-like cells expressing SOX10 and capable of differentiating into peripheral sensory neuron-like cells, such as nociceptor-like cells, are included among the embodiments of the present invention.

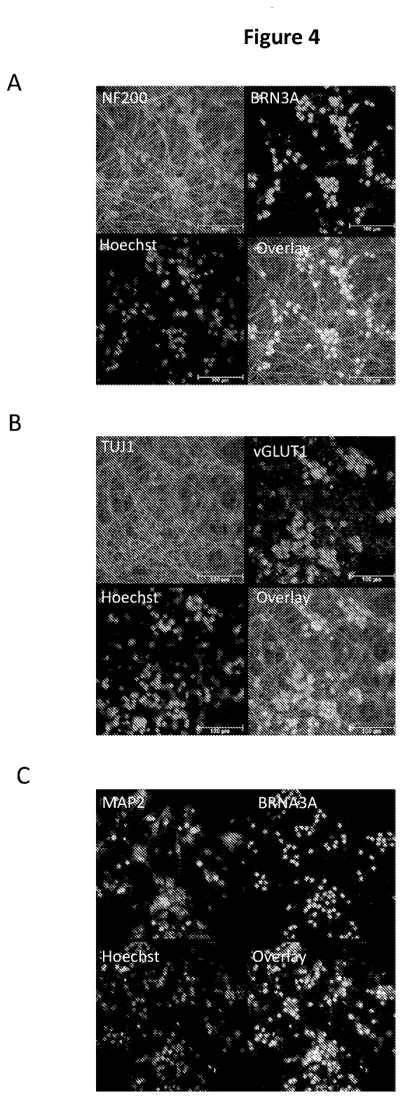

As discussed throughout the present document, some embodiments of the methods of the present invention produce nociceptor-like cells or their populations. Nociceptor-like cells, as discussed in this document, are cells exhibiting some properties of naturally occurring nociceptor cells, or sensory neurons possessing cell endings that initiate the sensation of pain. Nociceptors are specialized sensory neurons of the peripheral nervous system that respond to damaging stimuli (mechanical, thermal, chemical) and send signals to the spinal cord and the brain. The cell bodies of nociceptors are localized within dorsal root ganglia and because of their split axon (the projection to the periphery becoming free nerve endings and the central projection forming glutamatergic synaptic contacts with spinal cord neurons) they are described as pseudo-unipolar neurons. Nociceptor-like cells involved in the methods according to the embodiments of the present invention express at least one marker of naturally occurring nociceptor cells—BRN3A. Nociceptor-like cells involved in the methods according to the embodiments of the present invention can also express one or more of other markers expressed by naturally occurring nociceptors, such as ISL1, PRPH, DRGX, SLC17A6, or the ion channels and receptors discussed below. Naturally occurring nociceptors are glutamatergic neurons and express the vesicular glutamate transporter 1 (vGLUT1; FIG. 4B) Nociceptor-like cells involved in the methods according to the embodiments of the present invention can express or exhibit upregulated expression of one or more other markers found in natural nociceptors: NAV1.7, NAV1.8, NAV1.9, OPRM1 (mu opioid receptor), OPRK1 (kappa opioid receptor), OPRD1 (Delta opioid receptor), OPRL1 (opioid related nociceptin receptor 1). Naturally occurring nociceptors are pseudo-unipolar cells with a split axon that projects peripherally and centrally. Unlike many other neuronal cells, nociceptors don't develop dendrites in vivo. Accordingly, nociceptor-like cells involved in the methods according to the embodiments of the present invention can also be characterized by an absence of dendrites expressing detectable dendrite marker MAP2, and/or by ultrastructural analysis (for example, using electron microscopy) showing that the nociceptor-like cells lack dendrites. Compositions and kits including nociceptor-like cells characterized by the presence (such as the presence of one or more of ISL1, PRPH, DRGX, SLC17A6, vGLUT1, NAV1.7, NAV1.8, NAV1.9, OPRM1, OPRK1, OPRD1 or OPRL1) and/or absence (such as the absence of MAP2-positive dendrites) of the markers in this document are included among the embodiments of the present invention. The presence or absence of the markers, as applied to the embodiments of the preset invention, means detectable presence or absence of the markers as detected by applicable methods for detecting such markers, and may mean certain detectable or undetectable levels of such markers. In other words, the presence may mean the presence above a certain detectable level, while the absence may mean the absence below a certain detectable level and not necessarily zero detectable level. It is also to be understood that nociceptor-like cells may include a variety of cells on a continuum, with varying levels of presence or absence of certain detectable markers.

Compositions according to the embodiments of the present invention include in vitro or ex vivo compositions comprising at least one neural crest-like cell or at least one nociceptor-like cells. The cells included in such compositions can be vertebrate cells (meaning the cells originating from vertebrate PSCs), including mammalian cells (meaning the cells generated from mammalian PSCs) or human cells (meaning the cells generated from mammalian PSCs). The cells included in such compositions can be modified cells. The compositions can include pluralities of cells of the same or different type. For example, a plurality of cells can include one or more of a pluripotent stem cell, a multipotent stem cell, a progenitor cell, a differentiated cell, and a modified cell. A plurality of mammalian cells can be multiple cells, a cell culture, a cell aggregate, a spheroid or a tissue. At least one cell or a plurality of cells can be cryopreserved or thawed after cryopreservation. It is understood that some of the compositions according embodiments of the present invention can further comprise a culture medium, one or more additives, a vessel containing the culture medium, such as a culture flask, a culture dish, a tube or a reactor, and can also comprise a support or a scaffold for cells.

Using the described methods, compositions comprising various mixtures of pluripotent stem cells and other multipotent or differentiated cells can be produced. Such compositions are included among the embodiments of the present invention. In some embodiments, compositions comprising at least about 5 multipotent or differentiated cells for about every 95 pluripotent cells can be produced. In other embodiments, compositions comprising at least about 95 multipotent or differentiated cells for about every 5 pluripotent cells can be produced. Additionally, compositions comprising other ratios of multipotent or differentiated cells to pluripotent cells are contemplated. For example, compositions comprising at least about 1 multipotent or differentiated cell for about every 1,000,000 pluripotent cells, at least about 1 multipotent or differentiated cell for about every 100,000 pluripotent cells, at least about 1 multipotent or differentiated cell for about every 10,000 pluripotent cells, at least about 1 multipotent or differentiated cell for about every 1000 pluripotent cells, at least about 1 multipotent or differentiated cell for about every 500 pluripotent cells, at least about 1 multipotent or differentiated cell for about every 100 pluripotent cells, at least about 1 multipotent or differentiated cell for about every 10 pluripotent cells, at least about 1 multipotent or differentiated cell for about every 5 pluripotent cells, and up to about every 1 pluripotent cell and at least about 1,000,000 multipotent or differentiated cell for about every 1 pluripotent cell are contemplated. Some embodiments of the compositions can be cell cultures or cell populations comprising from at least about 5% multipotent or differentiated cell to at least about 99% multipotent or differentiated cells. In some embodiments the cell cultures or cell populations comprise mammalian cells. In preferred embodiments, the cell cultures or cell populations comprise human cells. For example, certain specific embodiments relate to cell cultures comprising human cells, wherein from at least about 5% to at least about 99% of the human cells are multipotent or differentiated cell. Other embodiments relate to cell cultures comprising human cells, wherein at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or greater than 99% of the human cells are multipotent or differentiated cells.

The progression of pluripotent cells to multipotent cells to further differentiated cells (for example, a progression from PSCs to neural crest-like cells, or a progression of neural crest-like cells to nociceptor-like cells) can be monitored by detecting the markers characteristic of the specific cell type. Identification of cell types related to the embodiments of the present invention can also be performed by detecting the markers characteristic of the specific cell type. For example, expression of certain markers can be detected. Expression of certain markers can be determined by detecting the presence or absence of the marker in cells, cell culture or cell population. Expression of certain markers can also be determined by measuring the level at which the marker is present in cells, cell culture or cell population. In some embodiments of the present invention, the expression of markers characteristic of neural crest-like cells, such as SOX10, can be determined. In some embodiments, the expression of one or more markers characteristic of nociceptor-like cells, such as BRN3A, TUJ1 (beta-III-Tubulin), Peripherin, ISL1, GGRP, TRPV1, NAV1.7, NAV1.8, NAV1.9, OPRM1, OPRMK1, PRD1, OPRL1 or NF200, can be determined. Quantitative, qualitative or semi-quantitative techniques can be used to measure marker expression. For example, marker expression can be detected and/or quantitated through the use of techniques detecting nucleic acids, such as PCR-based detection or RNA (for example, real-time reverse-transcriptase PCR), RNA sequencing (RNA-seq), or RNA detection by nucleic acid array-based techniques. In another example, immunochemistry can be used to detect and/or quantitate marker proteins. For example, the expression of a marker gene product can be detected by using antibodies specific for the marker gene product of interest using Western blotting, immunocytochemical characterization, flow cytometry analysis, etc. Various techniques of marker detection can be used in in conjunction to effectively and accurately characterize and identify cell types and determine both the amount and relative proportions of such markers in a subject cell type. The expression of certain markers can be determined by measuring the level at which the marker is present in the cells of the cell culture or cell population as compared to a standardized or normalized control marker. Identification and characterization of cells, cell cultures or cell population can be based on expression of a certain marker or different expression levels and patterns of more than one marker (including the presence or absence, the high or low expression, of one or more the markers). Also, certain markers can have transient expression, when the marker is exhibits higher expression during one or more stages of the processes described in this document and lower expression during other stage or stages.

Kits for cell, tissue or organ culture are included among embodiments of the present invention. A kit is a set of components, comprising at least some components for culturing cells, which can include single cells and groups of cells. A kit can contain one or more additives discussed in the corresponding section of this document. A kit may further contain one or more of the following: culture media configured to support at least one cell in vitro or ex vivo or one or more of culture media components; a vessel for holding the culture medium; a culture vessel, such as a flask, a dish, a plate (including a multi-well plater) or a reactor; or a support or scaffold for cell or tissue culture. A kit may contain one or more mammalian cells, such as human cells. Cells included in the kit can be one or more of: PSCs (including embryonic stem cells and/or induced pluripotent stem cells), neural crest-like cells or nociceptor-like cells. One or more cells can be provided in a frozen or non-frozen form (which can be a thawed form).

### Cryopreservation

Methods, compositions and kits that involve cryopreservation, including processes, tools and/or compositions related to cryopreservation, thawing and culturing of previously cryopreserved cells, cell populations or cell cultures are included among the embodiments of the present invention. Some compositions related to the preservation can include a cryopreservation medium used for the cryopreservation of cells or cell populations described in this document, such as neural crest-like cells and nociceptor-like cells. Some compositions can include a cryopreservation medium and one or more cells described in this document. For example, an embodiment of a composition can include one or more neural crest-like cells and a cryopreservation medium. In another example, a composition can include one or more nociceptor-like cells and a cryopreservation medium. The cryopreservation medium can be a liquid medium in which the cells are found prior to freezing and/or while in frozen state. Some examples of cryopreservation media are PSC Cryopreservation Kit (Thermo Fisher Scientific), FreezlS (Irving Scientifc), NutriFreez (Biological Industries USA), CryoStor, HypoThermosol, mFreSR, mFreSR-S, STEMdiff Neural Progenitor Freezing Medium (all from Stem Cell Technologies). Cryopreservation medium can contain one or more cryoprotectants, meaning compounds protecting cells from freezing damage. Cryoprotectants can be permeating or non-permeating. An example of a suitable permeating cryoprotectant, which is able to permeate cell membranes, is dimethyl sulfoxide (DMSO). Some examples of suitable non-permeating cryoprotectants are sucrose, glycerol, dextran, trehalose, percoll, polyethylene glycol, polyvinyl pyrrolidone, serum albumin, ficol, maltose and polyvinylalcohol (PVA). The cryopreservation medium can further contain one or more additives described in the section “Additives” of this document. For example, the cryopreservation medium can comprise one or more of Chroman-1 or its derivatives, Emricasan or its derivatives, trans-ISRIB or polyamines, at their respective effective combination. A combination of all four of the above additives can be referred to as “CEPT.”

Methods involving cryopreservation of cells, cell populations or cell cultures are included among the embodiments of the present invention. Such methods may include a step of contacting one more cells, such as neural crest-like cells or nociceptor-like cells with a cryopreservation medium. This may involve adding the cryopreservation medium to the one or more cells, or vice versa, and mixing the cells with the medium. In some embodiments, between 0.5 mL and 5 mL of cryopreservation medium may be added per one million cells, for example about 1 mL per million cells. However, it is envisaged that in certain embodiments, higher or lower amounts of cryopreservation medium can be used. In some embodiments, the cryopreservation medium may be added to the cells in step-wise increments of increasing concentration, which may reduce the risk of cellular osmotic shock associated with single-step addition. The temperature of the cryopreservation medium when added to the cells may range from about 15° C. to about 40° C. For example, the temperature of the cryopreservation medium added to the cells can be about 37° C. The contacting step of the present method may result in suspension of the cells in the cryopreservation medium, which can be referred to as “mixture.” The cells before the contacting step or the cell suspension after the contacting step may be provided in a container or a vessel. A container may have a volume between 1 mL and 50 mL, for example, it may be a tube of 15 mL.

Methods involving cryopreservation of cells may include a step of freezing a composition comprising one more cells, such as neural crest-like cells or nociceptor-like cells, and a cryopreservation medium, thereby obtaining a frozen or cryopreserved composition. A mixture of the cells and the cryopreservation medium can be equilibrated prior to freezing the mixture. During equilibration, water can be removed from the cells and replaced by the medium comprising the cryoprotectant, which enters into the cells after incubation of the cells with the cryopreservation medium. The equilibration time is limited to avoid damage to the cells. For example, the mixture can be equilibrated for a time period of between 10 seconds and 5 minutes, between 20 seconds and 1.5 minutes, or between 30 seconds to 1 minute. Before freezing, the mixture can be transferred to a freezing container or vessel, or remain in the same container in which the mixture already resided. Water can be removed from the cells and replaced by the medium comprising the cryoprotectant, which enters into the cells after incubation of the cells with the cryopreservation medium. The containers used for freezing typically provide for the stacking of tubes and can ensure that, by placing the container in a freezer, a fixed rate of cooling is achieved.

The freezing results in the cells in a cryogenic or cryopreserved state (which may simply be described as “frozen”), in which they can remain for periods of days, weeks, months or years, for retrieval when the cells are required. When needed, the cryopreserved cells are retrieved and thawed. Accordingly, methods involving cryopreservation can include a step of thawing a cryopreserved composition, more particularly under conditions that maintain cell viability. For example, a container containing the cryopreserved cells can be thawed in a bath of water, at a temperature of 42° C. or less, such as between 10° C. and 40° C., for example, at about 37° C. To improve the post-thaw cell viability, a thawing rate between about 10° C. and about 40° C. per minute, such as about 20° C. and about 40° C. per minute, for example, approximately 30° C. per minute may be used.

The described methods and/or method steps may lead to good viability of cryopreserved cells after thawing. As used herein, the term “viability” refers to the number of living cells based on the presence of DNA and an intact cell membrane system. Viability can be measured by various tests, such as a Trypan blue internalization test or by measuring propidium iodide uptake. The viability of the thawed cells after cryopreservation, such as thawed neural crest-like cells or thawed nociceptor cells can be at least 50%, at least 55%, at least 60%, at least 65%, at least 70%, at least 75%, at least 80%, at least 85%, at least 90%, or at least 95%. The cells may display a limited amount of necrosis and apoptosis after thawing. In particular embodiments, necrosis and/or apoptosis is observed in less than 25% of the cells, more particularly less than 15%, most particularly less than 10% of the cells. The methods described herein may further ensure that neural crest-like cells maintain their ability to differentiate into nociceptor-like cells. After thawing, the cryopreserved cells may be used for further culturing, differentiation (in the case of neural crest-like cells), therapeutic purposes, such as regenerative medicine, or other uses.

The following examples will serve to further illustrate the present invention without, at the same time, however, constituting any limitation thereof. On the contrary, it is to be clearly understood that resort may be had to various embodiments, modifications and equivalents thereof which, after reading the description herein, may suggest themselves to those skilled in the art without departing from the spirit of the invention.

### Example 1

### Differentiation of Human Pluripotent Stem Cells into Peripheral Sensory Neuron-Like Cells, Such as Nociceptor-Like Cells

Human pluripotent stem cells (hPSCs), including embryonic stem cells (ESCs) and induced pluripotent stem cells (iPSCs), were maintained and expanded in defined E8 medium. Human ESC lines were purchased from WiCell (Madison, Wis.) and iPSCs were generated by NIH. hPSCs were grown as attached monolayer cultures. At the start of the differentiation procedure, defined E8 medium was switched to E6 medium containing two small-molecule compounds: CHIR98014 (Selleckchem)—a compound capable of activating WNT signaling—at 1 μM final concentration in the medium; and A83-01 (Tocris)—a compound capable of inhibiting TGF-beta signaling, —at 2 μM final concentration at the medium. The day of the initial medium switch was termed “day 0.” At day three (approximately 72 hours) after the medium switch, the cells were enzymatically dissociated and plated into ultra-low attachment plates in order to initiate formation of nocispheres. During the nocisphere formation phase, the cells were cultured in E6 medium containing, in addition to 1 μM CHIR98014 and 2 μM A83-01, 1 μM DBZ (gamma-secretase inhibitor, Tocris) and 25 nM PD173074 (FGFR/VEGFR inhibitor, Tocris). E6 medium containing all the above compounds was changed approximately every 24 hours during the nocisphere formation phase, which continued until day 14 after the initial medium switch. In other words, nocisphere growth occurred during days 3-14 after the initial medium switch day (day 0), or for 11 days or approximate 244 hours. At day 14 after the initial medium switch (or approximately 336 hours after the initial medium switch), the formed nocispheres were enzymatically dissociated using the following process. The nocispheres were collected from the culture plates using a reversible 37 μm cell strainer (Stem Cell Technologies), by centrifugation or simply by sedimentation for 5 minutes in 15- or 50 mL conical tubes. The collected nocispheres were washed with phosphate buffered saline (PBS), and dissociated by incubating in TrypLE reagent (Thermo Fisher Scientific) at 37° C. for 10 minutes with constant shaking at 300 RPM. After the dissociation, the cells were collected by centrifugation at 300 g for 3 minutes, and the supernatant containing TrypLE reagent was discarded. The dissociated cells were either cryopreserved or plated. The dissociated cells were cryopreserved in DMEM/F12 medium (Thermo Fisher Scientific) supplemented with 10% DMSO (dimethyl sulfoxide) at 5-10 million cells per mL per tube using CoolCell® Cell Freezing Containers (Biocision) or plated on coated cell culture plates for further maturation. An exemplary cryopreservation procedure is described in Example 6. For plating of the dissociated cells, the medium was switched to DMEM/F12 medium (Thermo Fisher Scientific) supplemented with the following supplements: N2 supplement (100× solution, Thermo Fisher Scientific); B27 supplement (without Vitamin A) (50× solution, Thermo Fisher Scientific); the following growth factors at 25 ng/mL each—BDNF (Brain-Derived Neurotrophic Factor, R&D Systems), GDNF (Glial-Derived Neurotrophic Factor, R&D Systems), NGF (Nerve Growth Factor, R&D Systems), NT-3 (Neurotrophin-3, R&D Systems); and 1 μM PD0332991 (CDK4/6 inhibitor, Tocris). The plated dissociated nocisphere cells were cultured for up to 8 weeks, with the medium changed every 2-3 days. The above differentiation procedure is schematically illustrated in FIG. 1, with the small-molecule compounds used illustrated in FIG. 2.

### Example 2

### Immunocytochemical Characterization of Nociceptor-Like Cells Derived from hPSCs

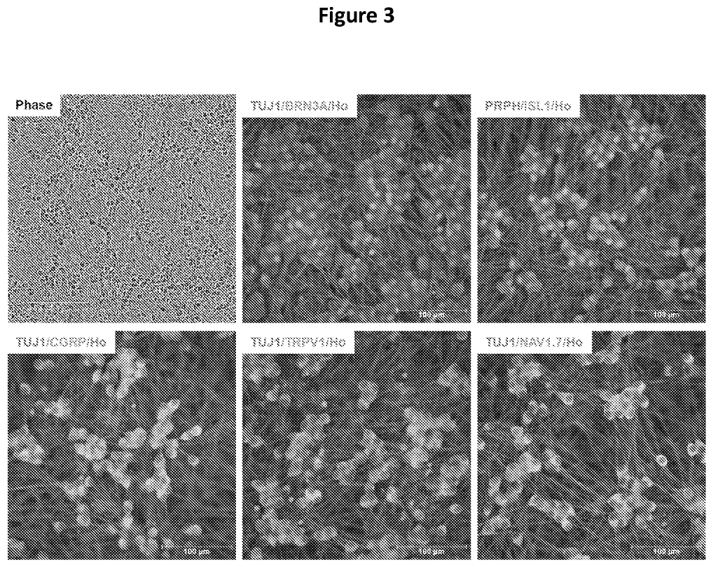

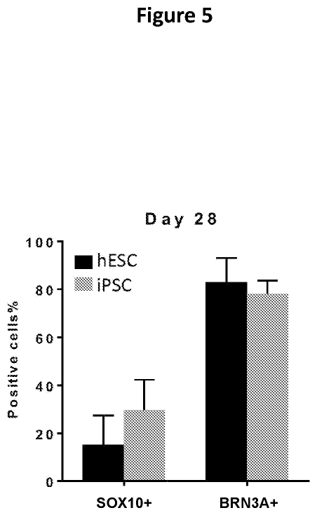

The cells produced according to the procedure described in Example 1 exhibited highly efficient differentiation into nociceptor-like cells, as illustrated by the figures discussed below. FIGS. 3 and 4 show representative images of plated cells taken at day 28 of culturing. FIG. 5 illustrates quantitative characterization of the plated cells at day 28 of culturing. Immunocytochemical characterization of the cultures derived from hPSCs confirmed differentiation of hPSCs into nociceptor-like cells expressing typical neuronal markers.

The cells shown in FIG. 3 were stained with combinations of antibodies (both monoclonal and polyclonal) specific for the following proteins: TUJ1 (neuronal marker); BRN3A (transcription factor typically expressed by nociceptors); peripherin (PRPH, marker for peripheral neurons); ISL1 (transcription factor expressed by nociceptors); CGRP (calcitonin-gene related protein, neuropeptide typically expressed by nociceptors); TRPV1 (vanilloid receptor 1, typically expressed by nociceptors); NAV1.7 (sodium channel, typically expressed by nociceptors). The label “Ho” indicates Hoechst counterstain, a dye commonly used for visualization of stained nuclei. The images included in FIG. 4 show individual immunostainings for NF200 (green), BRN3A (red) and Hoechst dye staining (blue). The cells shown in FIG. 4A were stained with monoclonal and polyclonal antibodies specific for NF200 (Neurofilament 200, a typical marker for visualizing neuronal cell bodies and axons) and the specific transcription factor BRN3A. Hoechst counterstain was used to visualize cell nuclei. The merged image in the bottom right corner of FIG. 4A shows all the immunostainings together. The images shown in FIG. 4A indicate that highly pure cultures of nociceptor-like cells co-expressing NF200 and BRN3A were generated using the procedure described in Example 1. The cells shown in FIG. 4B were stained with monoclonal and polyclonal antibodies specific for the neuronal marker TUJ1 (green), vesicular glutamate transporter 1 (vGLUT1; red) typically expressed by glutamatergic nociceptors, and Hoechst (blue). The cells shown in FIG. 4C were stained with monoclonal and polyclonal antibodies specific for MAP2 (marker for neuronal cell bodies and dendrites; green), BRN3A (red) and Hoechst dye (blue). Poor staining of neuronal processes by MAP2 indicated the absence of dendrites, which was consistent with the appropriate anatomy of nociceptors as pseudo-unipolar neurons with a split axon projecting to the periphery and the spinal cord but absence of dendrites, unlike other neurons (for example, cortical neurons develop elaborate dendritic trees). To generate FIG. 5, two hPSC lines, ESCs and iPSCs, were differentiated into nociceptor-like cells according to the procedure described in Example 1. At day 28 (approximately 672 hours) after the initial media switch, the cells were stained for SOX10 (marker for neural crest stem cells) and BRN3A (marker for nociceptors). The staining was quantified using ImageJ software (National Institutes of Health). The quantification indicated that the levels of the above markers were consistent with the presence of SOX10-expressing neural crest-like cells and BRN3A-positive nociceptor-like cells. The combined results of the staining and the quantification, discussed above, also indicated that only neural crest-like cells and nociceptor-like were present in the cultures generated by the procedure described in Example 1.

### Example 3

### Time-Course Gene Expression Profiling

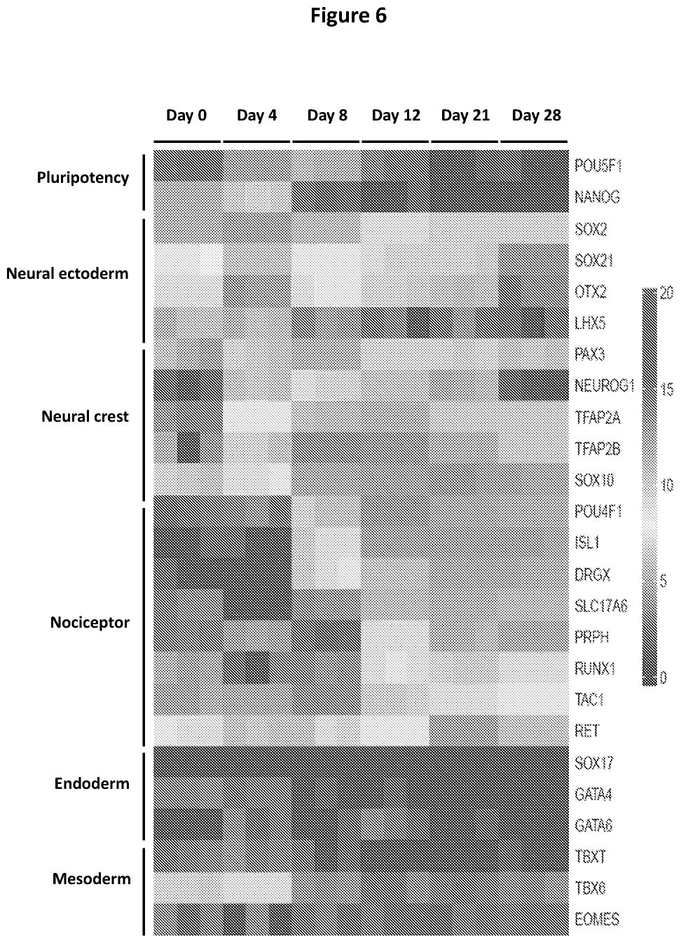

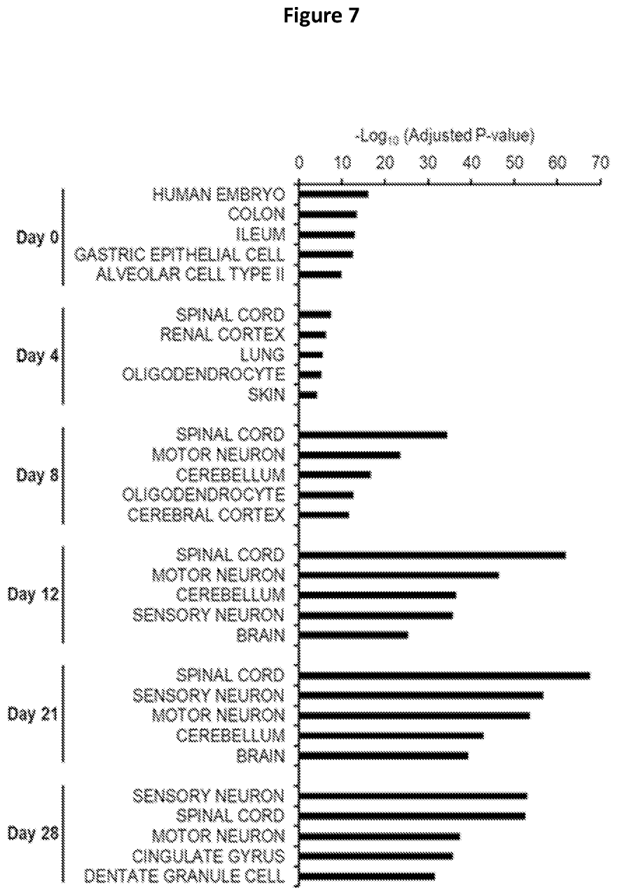

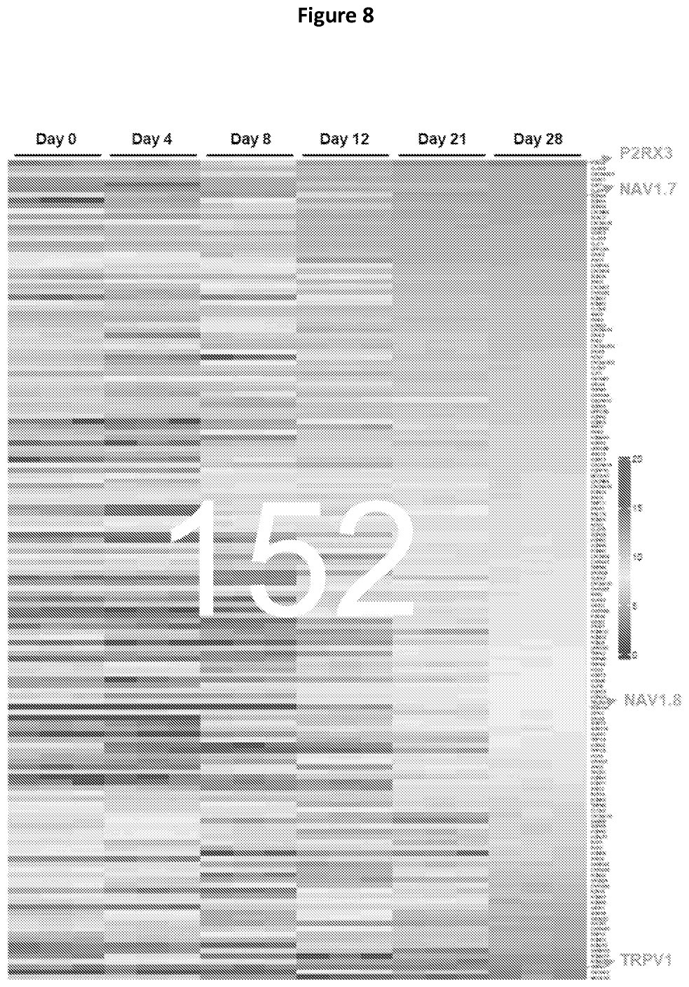

The cultures produced according to the procedure described in Example 1 were characterized by time-course gene expression profiling accomplished by RNA-seq analysis, as illustrated by the figures discussed below. Time-course RNA-Seq analysis of the cultures derived from iPSCs confirmed differentiation of iPSCs into nociceptor-like cells expressing typical neuronal markers, such as transcription factors, neuropeptides, and ion channels. FIG. 6 illustrates the results of systematic analysis of the time course of gene expression over days 0-28 of the procedure described in Example 1, which demonstrated step-wise and controlled differentiation of iPSCs into neural ectoderm-like cells, neural crest-like cells and nociceptor-like cells. The genes indicative of non-neural cell lineages, such as endoderm and mesoderm, were absent from the differentiated cultures, thus indicating high efficiency of differentiation. FIG. 7 illustrates the results of time-course gene expression profiling by RNA-seq of nociceptor differentiation and comparison of the results to those available in ARCHS⁴ human tissue RNA-seq database (Lachmann et al. “Massive mining of publicly available RNA-seq data from human and mouse” Nature Communications 9:1366 (2018)) available online through Mount Sinai Center for Bioinformatics, Icahn School of Medicine at Mount Sinai (New York, N.Y., USA). For gene ontology analysis, a web-based tool, EnrichR (available online through Icahn School of Medicine at Mount Sinai) was used to compare the top 200 genes upregulated in the culture at each tested time point and compared to the data found in ARCHS⁴. The top five enriched categories at each timepoint were plotted. FIG. 8 illustrates the results of comprehensive analysis by RNA-seq of the ion channels expressed by nociceptor-like cells generated by the procedure described in Example 1. Remarkably, 152 ion channel genes, including NAV1.8 and opioid receptors, were expressed by the nociceptor-like cells at day 28 of the procedure. Such comprehensive ion channel expression by cultured stem cell-derived nociceptor-like cells has not been previously achieved.

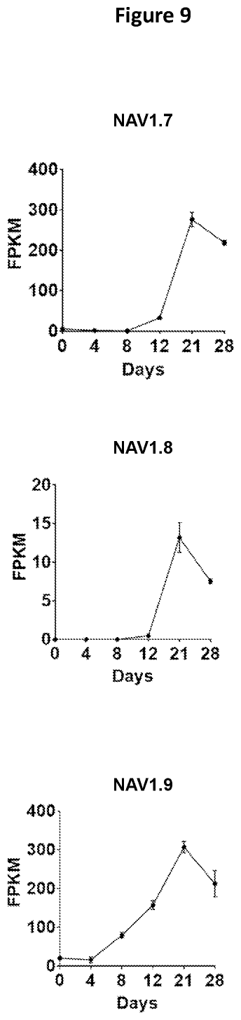

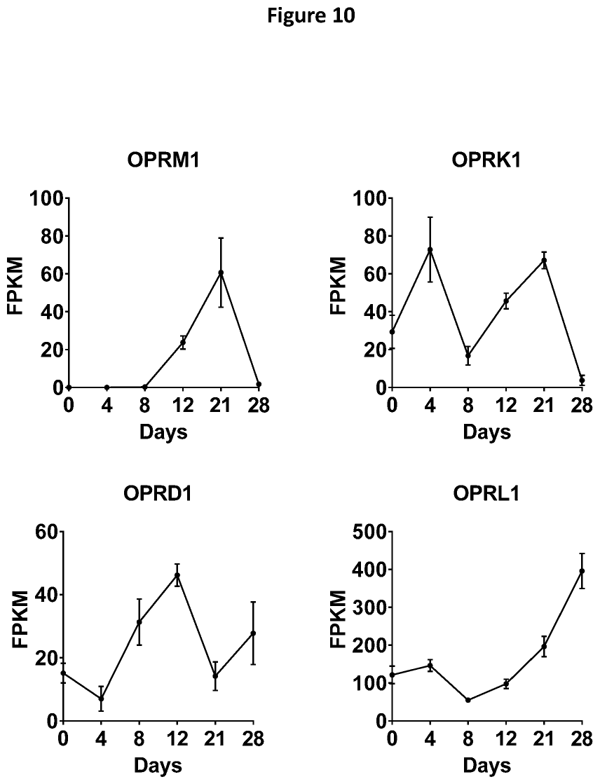

FIGS. 9 and 10 illustrate the results of time-course gene expression profiling by RNA-seq in the nociceptor-like cells generated by the procedure described in Example 1. Plotted on the X-axis of each line plot shown in FIGS. 9 and 10 are days of the procedure, as described in Example 1. Plotted on the Y-axis of each line plot shown in FIGS. 9 and 10 is a value of Fragments Per Kilobase of Transcript per Million Mapped Reads (FPKM). FPKM value indicates the relative expression level of a transcript in RNA-seq analysis. FIG. 9 illustrates the results of time-course gene expression profiling by RNA-seq of important sodium channels in the nociceptor-like cells generated by the procedure described in Example 1. The profiling of the sodium channels demonstrated the upregulation of NAV1.7, NAV1.8 and NAV1.9 genes during cell differentiation. Previously available procedures for production of nociceptor-like cells in culture from stem cells did not achieve the expression of the above three critical sodium channels. FIG. 10 illustrates the results of time-course gene expression profiling by RNA-seq analysis of opioid receptors in the cultures being differentiated from iPSCs following the procedure described in Example 1. The results of the opioid receptor profiling demonstrated that important opioid receptors (OPRM1—mu opioid receptor; OPRK1—kappa opioid receptor; OPRD1—delta opioid receptor) and the opioid related nociceptin receptor 1 (OPRL1) were expressed and regulated in cultured cells over the course of the procedure described in Example 1.

### Example 4

### Functional Analysis of Nociceptor-Like Cells Derived from iPSCs

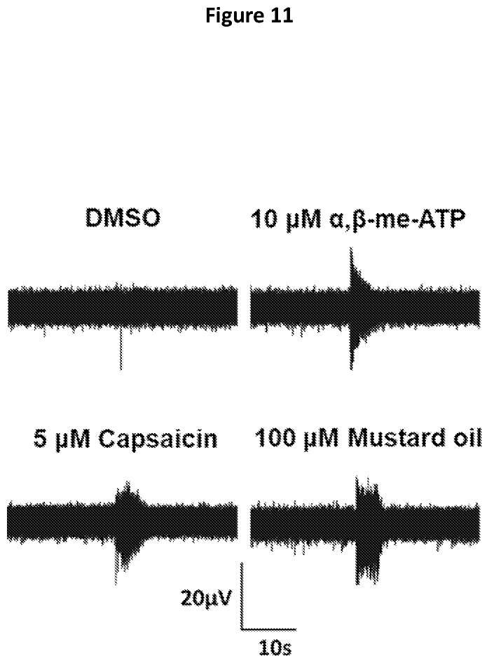

Functional analysis of nociceptors derived from iPSCs according to the procedure described in Example 1 was performed. Multi-electrode arrays were used for the electrophysiology characterization experiments. FIG. 11 illustrates the results of an electrophysiology experiment (multi-electrode arrays using Maestro Pro, Axion Biosystems) demonstrating that iPSC-derived nociceptor-like cells were stimulated by specific ligands. As illustrated in FIG. 11, application of 10 μM α,β-me-ATP, 5 μM capsaicin (a known activator of the vanilloid receptor TRPV1, a heat-sensitive cation channel on nociceptor terminals), and 100 μM mustard oil (allyl isothiocyanate; a plant-derived irritant that activates transient receptor potential (TRP) family receptors and is widely used to induce pain and inflammation in pain research) elicited a specific response by increasing the frequency of firing action potentials.

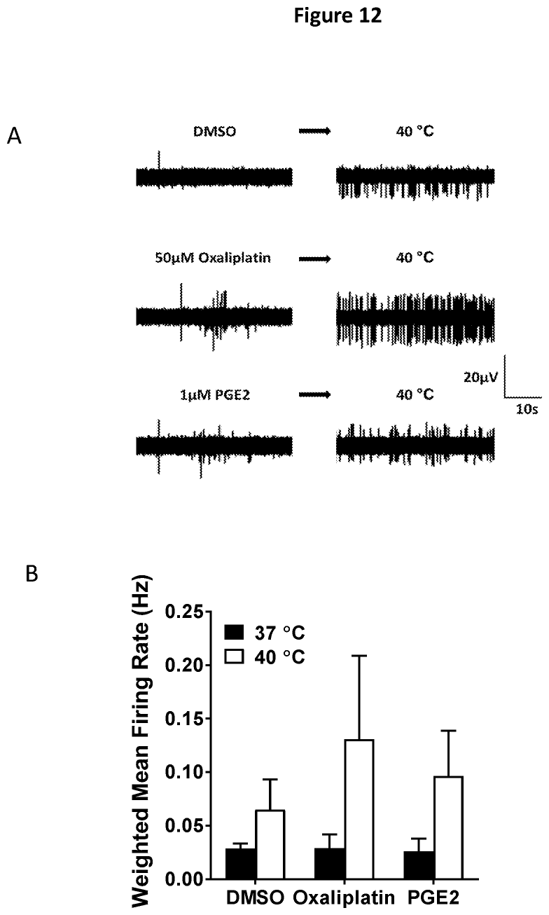

FIGS. 12A and 12B illustrate the results of an electrophysiology experiment (multi-electrode array using Maestro Pro, Axion Biosystems) showing that iPSC-derived nociceptor-like cells were sensitized in response to treatment with oxaliplatin and prostaglandin E2 (PGE2). Oxaliplatin is a widely used chemotherapeutic drug that can damage sensory neurons of the peripheral nervous system and lead to peripheral neuropathy. PGE2 is used to model inflammatory pain. At day 28 of the procedure described in Example 1, the iPSC-derived nociceptor-like cells were recorded at 37° C. for 10 minutes for baseline recording, and then pre-treated with 0.1% DMSO, 50 μM Oxaliplatin or 1 μM PGE2 for 15 minutes at 37° C. The temperature was then increased to 40° C. to stimulate the cells, and recording was performed for another 10 minutes. The above-described experiments showed that iPSC-derived nociceptor-like cells were sensitized in response to treatment with oxaliplatin and PGE2, demonstrating that iPSC-derived nociceptor-like cells were useful as an in vitro model for investigating the pathological changes that lead to chemotherapy- or inflammation-induced pain.

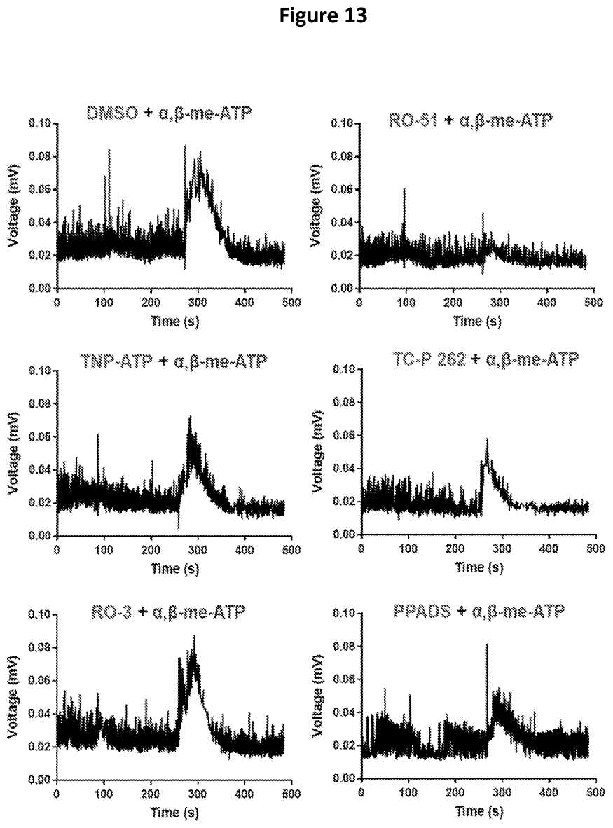

FIG. 13 illustrates the results of an electrophysiology experiment (multi-electrode array using Maestro Pro, Axion Biosystems) demonstrating that iPSC-derived nociceptor-like cells were stimulated by application of 10 μM α,β-me-ATP, which is a known agonist for P2RX3 purinergic receptors. At day 28 of the procedure described in Example 1, the iPSC-derived nociceptor-like cells were pre-treated with 0.1% DMSO or 10 μM of each known antagonist of purinergic receptor P2RX3 for 30 minutes at 37° C. The cells were then stimulated by 10 μM α,β-me-ATP, and recordings were performed using multi-electrode. The recordings showed the differential response indicating that RO-51 was the most potent inhibitor of P2RX3 receptors. The above results demonstrated that that iPSC-derived nociceptor-like cells were useful as an in vitro model for drug discovery and drug testing.

### Example 5

### Automated Procedure

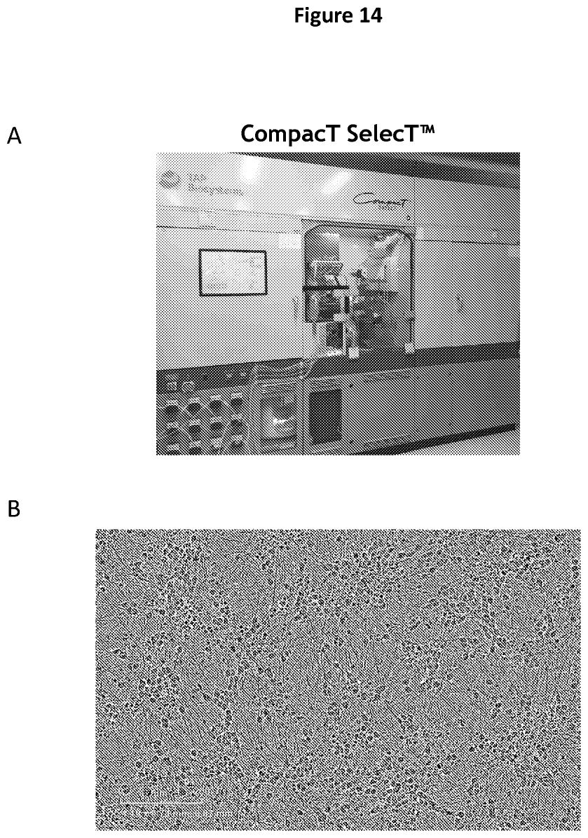

The procedure described in Example 1 was used as a basis for an automated procedure by using the CompacT SelecT® system (Sartorius, Wilmington, USA) illustrated in FIG. 14A. Highly efficient, standardized and scalable production of nociceptor-like cells from iPSCs was achieved using the automated procedure. FIG. 14B shows a representative microscopic image of nociceptor-like cells produced by automated differentiation at day 21 after the start of the automated procedure. The image shown FIG. 14B shows the nociceptor-like cells forming a highly dense network of neuronal processes.

### Example 6

### Cryopreservation

Nocispheres generated according to the procedure described in Example 1 are collected from the culture flasks at day 14 using a reversible 37 μm cell strainer (Stem Cell Technologies), washed with PBS, and dissociated by incubating in TrypLE reagent (Thermo Fisher Scientific) at 37° C. for 10 minutes with constant shaking at 300 RPM. After collecting the dissociated cells by centrifugation at 300 g for 3 minutes (the supernatant containing TrypLE is discarded), the collected cells are resuspended and cryopreserved in DMEM/F12 medium (Thermo Fisher Scientific) supplemented with 10% DMSO at 5-10 million cells per mL per tube using CoolCell® Cell Freezing Containers (Biocision) or other appropriate cryopreservation methods. A small molecule cocktail can be included in a cryopreservation medium to improve cell survival post-cryopreservation. The small-molecule cocktail can include 50 nM Chroman 1, 5 μM Emricasan, Polyamines (40 ng/mL Putrescine, 4.5 ng/mL Spermidine, 8 ng/mL Spermine) and 0.7 μM Trans-ISRIB. The concentrations listed are final concentrations in the cryopreservation medium.

1. A method of producing, in culture, cells capable of differentiating into nociceptor-like cells, the method comprising:
incubating for approximately 24-144 hours attached monolayer cultures of vertebrate pluripotent stem cells in a first medium comprising an effective amount or concentration of at least one first compound capable of activating WNT signaling and an effective amount or concentration of at least one second compound capable of inhibiting TGF-beta signaling;
dissociating the incubated cells; and,
culturing the dissociated cells for 168-432 hours in a second medium comprising an effective amount or concentration of at least one third compound capable of activating WNT signaling, an effective amount or concentration of at least one fourth compound capable of inhibiting TGF-beta signaling, an effective amount or concentration of at least one fifth compound capable of inhibiting Notch pathway, and an effective amount or concentration of at least one sixth compound capable of inhibiting one or more of EGF, VEGF and MAP kinase signaling, thereby generating one or more nocispheres comprising the cells capable of differentiating into the nociceptor-like cells.

2. The method of claim 1, wherein the cells capable of differentiating into nociceptor-like cells are neural crest-like cells.

3. The method of claim 1 or 2, wherein the cells capable of differentiating into nociceptor-like cells detectably express SOX10.

4. The method of any one of claims 1-3, wherein the one or more nocispheres further comprise the nociceptor-like cells.

5. The method of any one of claims 1-4, wherein the nociceptor-like cells detectably express BRN3A.

6. The method of any one of claims 1-5, wherein the vertebrate pluripotent stem cells are induced pluripotent stem cells or embryonic pluripotent stem cells.

7. The method of any one of claims 1-6, wherein the vertebrate pluripotent stem cells are human pluripotent stem cells.

8. The method of any one claims 1-7, wherein the first medium is a defined medium and the second medium is a defined medium, and wherein the first medium is same or different from the second medium.

9. The method of any one of claims 1-8, wherein the first medium is E6, DMEM-F12 or Knockout-DMEM/F12.

10. The method of any one of claims 1-9, wherein the second medium is E6, DMEM-F12 or Knockout-DMEM/F12

11. The method of any one of claims 1-10, wherein the first medium and/or the second medium is not supplemented with additives activating or inhibiting bone morphogenic protein (BMP) protein pathways.

12. The method of any one of claims 1-10, wherein the first medium and/or the second medium is not supplemented with Bone Morphogenic Protein 4 (BMP4).

13. The method of any one of claims 1-12, wherein the at least one first compound is CHIR98014 or CHIR99021, or a combination thereof.

14. The method of claim 13, wherein the effective concentration of CHIR98014 or CHIR99021 is 20 nM-20 μM.

15. The method of any one of claims 1-14, wherein the at least one second compound is A83-01 or SB431542, or a combination thereof.

16. The method of claim 15, wherein the effective concentration of A83-01 is 20 nM-20 μM, and wherein the effective concentration of SB431542 is 20 nM-40 μM.

17. The method of any one of claims 1-16, wherein the at least one third compound is CHIR98014 or CHIR99021, or a combination thereof.

18. The method of claim 17, wherein the effective concentration of CHIR98014 or CHIR99021 is 20 nM-20 μM.

19. The method of any one of claims 1-18, wherein the at least one fourth compound is A83-01 or SB431542, or a combination thereof.

20. The method of claim 19, wherein the effective concentration of A83-01 is 20 nM-20 μM, and wherein the effective concentration of SB431542 is 20 nM-40 μM.

21. The method of any one of claims 1-20, wherein the at least one fifth compound is DBZ, DAPT, LY411575 or LY 3039478, or a combination of two or more thereof.

22. The method of claim 21, wherein the effective concentration of DBZ is 20 nM-20 μM, the effective concentration of DAPT is 5 nM-50 μM, the effective concentration of LY411575 is 2 nM-20 μM, and the effective concentration of LY3039478 is 2 nM-20 μM.

23. The method of any one of claims 1-22, wherein the at least one sixth compound is PD173074 or SU5402, or a combination thereof.

24. The method of claim 23, wherein the effective concentration of PD173074 or SU5402 is 2 nM-20 μM.

25. The method of any one of claims 1-24, wherein the second medium further comprises an effective amount or concentration of at least one seventh compound that is a CDK4/6 inhibitor.

26. The method of claim 25, wherein the CDK4/6 inhibitor is PD0332991.

27. The method of claim 26, wherein the effective concentration of PD0332991 is 2 nM-20 μM.

28. The method of any one of claims 1-27, wherein the culturing of the dissociated cells comprises changing the second medium approximately every 12-36 hours.

29. The method of any one of claims 1-28, further comprising dissociating the one or more nocispheres, thereby generating dissociated nocisphere cells.

30. The method of any one of claims 1-29, further comprising cryopreserving one or more of the cells capable of differentiating into the nociceptor-like cells, the one or more nocispheres, or the dissociated nocisphere cells.

31. A method of culturing nociceptor-like cells, comprising:
performing the method of claim 29;
growing in culture the dissociated nocisphere cells under conditions promoting differentiation of the nociceptor-like cells.

32. The method of claim 31, wherein the growing is conducted for at least approximately 168 hours or for 168-336 hours

33. The method of claim 31 or 32, wherein the conditions comprise the presence of N2 supplement and B27 supplement.

34. The method of claim 33, wherein the conditions further comprise the presence of one or more of BDNF, GDNF, NGF or NT-3.

35. The method of claim 33 or 34, wherein the conditions further comprise the presence of one or more of an effective amount or concentration of at least one eighth compound capable of inhibiting Notch pathway, an effective amount or concentration of at least one ninth compound capable of inhibiting one or more of EGF, VEGF and MAP kinase signaling or an effective amount or concentration of at least one ninth compound that is a CDK4/6 inhibitor.

36. The method of claim 35, wherein the at least one eighth compound is DBZ, DAPT, LY411575 or LY 3039478, or a combination of two or more thereof.

37. The method of claim 36, wherein the effective concentration of DBZ is 20 nM-20 μM, the effective concentration of DAPT is 5 nM-50 μM, the effective concentration of LY411575 is 2 nM-20 μM, and the effective concentration of LY3039478 is 2 nM-20 μM.

38. The method of claim 35, wherein the at least one sixth compound is PD173074 or SU5402, or a combination thereof.

39. The method of claim 38, wherein the effective concentration of PD173074 or SU5402 is 2 nM-20 μM.

40. The method of claim 35, wherein the CDK4/6 inhibitor is PD0332991.

41. The method of claim 40, wherein the effective concentration of PD0332991 is 2 nM-20 μM.

42. The method of any one of claims 31-41, wherein the conditions comprise the absence of supplementation with additives activating or inhibiting Bone Morphogenic Protein (BMP) protein pathways.

43. The method of any one of claims 31-41, wherein the conditions comprise the absence of supplementation with Bone Morphogenic Protein 4 (BMP4).

44. The method of any one of claims 31-43, wherein the conditions comprise culturing in DMEM/F12 medium, Neurobasal medium or BrainPhys medium.

45. The method of any one of claims 31-44, wherein the nociceptor-like cells detectably express one or more of TUJ1, Peripherin, ISL1, GGRP, TRPV1, NAV1.7, NAV1.8, NAV1.9, OPRM1, OPRMK1, OPRD1, OPRL1 or NF200.

46. The method of any one of claims 31-45, wherein the nociceptor-like cells detectably express NAV1.8, OPRM1, OPRMK1 and OPRD1.

47. The method of any one of claims 31-46, wherein the nociceptor-like cells lack dendrites detectably expressing MAP2.

48. The method of any one of claims 1-47, further comprising, after culturing the dissociated cells, cryopreserving the one or more nocispheres and thawing of the cryopreserved one or more nocispheres.

49. The method of any one of claims 1-47, further comprising, after dissociating the one or more nocispheres, cryopreserving the dissociated nocisphere cells and thawing of the dissociated nocisphere cells.

50. The method of any one of claims 31-49, further comprising cryopreserving the nociceptor-like cells.

51. The method of any one of claims 48-50, wherein one or more of cryopreserving the one or more nocispheres, cryopreserving the dissociated nocisphere cells or cryopreserving of the nociceptor-like cells is conducted in a cryopreservation medium comprising Chroman 1 and/or a derivative thereof, Emricasan and/or the derivative thereof, trans-ISRIB and polyamines comprising putrescine, spermine and spermidine.

52. The method of claim 51, wherein Chroman 1 and/or the derivative thereof is at a concentration of about 4 nM to about 40 μM, about 10 nM to about 20 μM, about 20 nM to about 10 μM or about 30 nM to about 500 nM, wherein Emricasan and/or the derivative thereof at a concentration of about 100 nM to about 40 μM, about 200 nM to about 30 μM, about 300 nM to about 20 μM, wherein trans-ISRIB at a concentration of about 50 nM to about 6.25 μM, about 100 nM to about 6.25 μM, or about 200 nM to about 6.25 μM, wherein putrescine, spermine and spermidine is each at a concentration of about 0.5 μM to 1 mM.

53. The method of any one of claims 1-52, wherein one or more steps of the method is performed by an automated system.

54. A composition comprising at least one cultured nociceptor-like cell detectably expressing one or more of BRN3A, TUJ1, Peripherin, I1, GGRP, TRPV1, NAV1.7, NAV1.8, NAV1.9, OPRM1, OPRMK1, OPRD1, OPRL1 or NF200.

55. The composition of claim 54, wherein the at least one cultured nociceptor-like cell detectably expresses NAV1.8, OPRM1, OPRK1 and OPRD1.

56. The composition of claim 54 or 55, wherein the at least one cultured nociceptor-like cell lacks dendrites detectably expressing MAP2.

57. The composition of any one of claims 55-56, wherein the at least one cultured nociceptor-like cell is or has been cryopreserved.

58. The composition of claim 57, wherein the at least one cultured nociceptor-like cell is or was cryopreserved in a cryopreservation medium comprising Chroman 1 and/or a derivative thereof, Emricasan and/or the derivative thereof, trans-ISRIB and polyamines comprising putrescine, spermine and spermidine.

59. The composition of claim 58, wherein Chroman 1 and/or the derivative thereof is at a concentration of about 4 nM to about 40 μM, about 10 nM to about 20 μM, about 20 nM to about 10 μM or about 30 nm to about 500 nM, wherein Emricasan and/or the derivative thereof at a concentration of about 100 nM to about 40 μM, about 200 nM to about 30 μM, about 300 nM to about 20 μM, wherein trans-ISRIB at a concentration of about 50 nM to about 6.25 μM, about 100 nM to about 6.25 μM, or about 200 nM to about 6.25 μM, wherein putrescine, spermine and spermidine is each at a concentration of about 0.5 μM to 1 mM.

60. A cell culture comprising the composition of any one of claims 55-56.

61. The cell culture of claim 60, further comprising a culture medium comprising Chroman 1 and/or a derivative thereof, Emricasan and/or the derivative thereof, trans-ISRIB and polyamines comprising putrescine, spermine and spermidine.

62. The cell culture of claim 61, wherein Chroman 1 and/or the derivative thereof is at a concentration of about 4 nM to about 40 μM, about 10 nM to about 20 μM, about 20 nM to about 10 μM or about 30 nM to about 500 nM, wherein Emricasan and/or the derivative thereof at a concentration of about 100 nM to about 40 μM, about 200 nM to about 30 μM, about 300 nM to about 20 μM, wherein trans-ISRIB at a concentration of about 50 nM to about 6.25 μM, about 100 nM to about 6.25 μM, or about 200 nM to about 6.25 μM, wherein putrescine, spermine and spermidine is each at a concentration of about 0.5 μM to 1 mM.

63. The cell culture of any one of claims 60-62, wherein the cell culture is grown from previously cryopreserved cells.

64. The cell culture of claim 63, wherein the previously cryopreserved cells were cryopreserved in a cryopreservation medium comprising Chroman 1 and/or a derivative thereof, Emricasan and/or the derivative thereof, trans-ISRIB and polyamines comprising putrescine, spermine and spermidine.

65. The cell culture of claim 64, wherein Chroman 1 and/or the derivative thereof is at a concentration of about 4 nM to about 40 about 10 nM to about 20 about 20 nM to about 10 μM or about 30 nm to about 500 nM, wherein Emricasan and/or the derivative thereof at a concentration of about 100 nM to about 40 about 200 nM to about 30 about 300 nM to about 20 wherein trans-ISRIB at a concentration of about 50 nM to about 6.25 about 100 nM to about 6.25 or about 200 nM to about 6.25 wherein putrescine, spermine and spermidine is each at a concentration of about 0.5 μM to 1 mM.

66. A composition comprising at least one cultured neural crest-like cell.

67. The composition of claim 66, wherein the at least one cultured neural crest-like cell is capable of differentiating into at least one nociceptor-like cell.

68. The composition of claim 66 or 67, wherein the at least one cultured neural crest-like cell detectably expresses SOX10.

69. The composition of any one of claims 66-68, wherein the composition further comprises at least one cultured nociceptor-like cell detectably expressing one or more of BRN3A, TUJ1, Peripherin, I1, GGRP, TRPV1, NAV1.7, NAV1.8, NAV1.9, OPRM1, OPRMK1, OPRD1, OPRL1 or NF200.

70. The composition of claim 69, wherein the at least one cultured nociceptor-like cell detectably expresses NAV1.8, OPRM1, OPRK1 and OPRD1.

71. The composition of claim 69 or 70, wherein the at least one cultured nociceptor-like cell lacks dendrites detectably expressing MAP2.

72. The composition of any one of claims 66-71, wherein the composition is or comprises one or more nocispheres.

73. The composition of any one of claims 66-72, wherein the composition comprises dissociated nocisphere cells.

74. The composition of any one of claims 66-73, wherein the composition is or was cryopreserved.

75. The composition of claim 74, wherein the composition is or was cryopreserved in a cryopreservation medium comprising Chroman 1 and/or a derivative thereof, Emricasan and/or the derivative thereof, trans-ISRIB and polyamines comprising putrescine, spermine and spermidine.

76. The composition of claim 75, wherein Chroman 1 and/or the derivative thereof is at a concentration of about 4 nM to about 40 about 10 nM to about 20 about 20 nM to about 10 μM or about 30 nM to about 500 nM, wherein Emricasan and/or the derivative thereof at a concentration of about 100 nM to about 40 about 200 nM to about 30 about 300 nM to about 20 wherein trans-ISRIB at a concentration of about 50 nM to about 6.25 about 100 nM to about 6.25 or about 200 nM to about 6.25 wherein putrescine, spermine and spermidine is each at a concentration of about 0.5 μM to 1 mM.

77. A cell culture comprising the composition of any one of claims 66-73.

78. The cell culture of claim 77, further comprising a culture medium comprising Chroman 1 and/or a derivative thereof, Emricasan and/or the derivative thereof, trans-ISRIB and polyamines comprising putrescine, spermine and spermidine.

79. The cell culture of claim 78, wherein Chroman 1 and/or the derivative thereof is at a concentration of about 4 nM to about 40 about 10 nM to about 20 about 20 nM to about 10 μM or about 30 nm to about 500 nM, wherein Emricasan and/or the derivative thereof at a concentration of about 100 nM to about 40 about 200 nM to about 30 about 300 nM to about 20 wherein trans-ISRIB at a concentration of about 50 nM to about 6.25 about 100 nM to about 6.25 μM, or about 200 nM to about 6.25 μM, wherein putrescine, spermine and spermidine is each at a concentration of about 0.5 μM to 1 mM.

80. The cell culture of any one of claims 77-79, wherein the cell culture is grown from previously cryopreserved cells.

81. The cell culture of claim 80, wherein the previously cryopreserved cells were cryopreserved in a cryopreservation medium comprising Chroman 1 and/or a derivative thereof, Emricasan and/or the derivative thereof, trans-ISRIB and polyamines comprising putrescine, spermine and spermidine.

82. The cell culture of claim 81, wherein Chroman 1 and/or the derivative thereof is at a concentration of about 4 nM to about 40 μM, about 10 nM to about 20 μM, about 20 nM to about 10 μM or about 30 nm to about 500 nM, wherein Emricasan and/or the derivative thereof at a concentration of about 100 nM to about 40 μM, about 200 nM to about 30 μM, about 300 nM to about 20 μM, wherein trans-ISRIB at a concentration of about 50 nM to about 6.25 μM, about 100 nM to about 6.25 μM, or about 200 nM to about 6.25 μM, wherein putrescine, spermine and spermidine is each at a concentration of about 0.5 μM to 1 mM.
